(19) United States
(12) Patent Application Publication
(10) Pub. No.: US 2026/0284082 A1
(43) Pub. Date: Sep. 24, 2026

(54) TARGETING THE PANX1-P2Y2R-TRPV4 PATHWAY TO TREAT LUNG ISCHEMIA-REPERFUSION INJURY

(71) Applicant: UNIVERSITY OF VIRGINIA PATENT FOUNDATION, Charlottesville, VA (US)

(72) Inventors: Swapnil Sonkusare, Charlottesville, VA (US); Victor Laubach, Waynesboro, VA (US); Huy Ta, Charlottesville, VA (US)

(21) Appl. No.: 19/474,695

(22) PCT Filed: Apr. 12, 2024
(86) PCT No.: PCT/US2024/024458

§ 371 Date: Oct. 10, 2025

Related U.S. Application Data
(60) Provisional application No. 63/496,215, filed on Apr. 14, 2023.

(51) Int. Cl. A61K 31/585; A61K 9/00; A61K 31/4709; A61K 31/506; A61P 11/00

(52) U.S. Cl. CPC A61K 31/585; A61K 9/0019; A61K 31/4709; A61K 31/506; A61P 11/00

(57) ABSTRACT

Provided herein are methods for treating lung ischemia-reperfusion injury (IRI) by targeting the purinergic P2Y2 receptor (P2Y2R) and other members of the Panx1-P2Y2R-TRPV4 signaling pathway.

### CROSS-REFERENCE TO RELATED APPLICATIONS

This application claims the benefit of and priority to U.S. Provisional Application No. 63/496,215 filed on Apr. 14, 2023, the content of which is incorporated by reference in its entirety.

### STATEMENT REGARDING FEDERALLY SPONSORED RESEARCH

This invention was made with government support under HL157407 awarded by the National Institutes of Health. The government has certain rights in the invention.

### REFERENCE TO AN ELECTRONIC SEQUENCE LISTING

The contents of the electronic sequence listing (182506.00010.xml: Size: 8,922 bytes; and Date of Creation: Apr. 12, 2024) is herein incorporated by reference in its entirety.

### BACKGROUND OF THE INVENTION

Lung ischemia-reperfusion injury (IRI) is lung tissue damage caused when blood supply returns to the lung after a period of lack of oxygen due to restriction in blood supply.

When circulation is restored, instead of restoration of normal tissue function, inflammation and oxidative damage occurs. IR is a common occurrence after lung transplantation. The robust inflammation, increased vascular permeability, and lung edema, is the major cause of primary graft dysfunction after lung transplantation. Strategies to prevent or reduce the occurrence of IRI, and thereby increase the success rate of lung transplantation, are needed.

### SUMMARY

In a first aspect, provided herein is a method for treating lung ischemia-reperfusion injury (IRI) in a subject in need thereof, the method comprising administering to the subject a P2Y2 receptor (P2Y2R) inhibitor. In embodiments, the P2Y2R inhibitor inhibits at least one of P2Y2R expression and activity. In embodiments, the P2Y2R inhibitor inhibits P2Y2R activity. In embodiments, the P2Y2R inhibitor is a selected from a compound, a protein, and an inhibitory nucleic acid. In embodiments, the P2Y2R inhibitor is a compound. In embodiments, the compound is AR-C 118925XX (ARC) or a pharmaceutically acceptable salt thereof. In embodiments, the inhibitory nucleic acid is selected from a dsRNA, a siRNA, a shRNA, a miRNA, an ami-RNA, an antisense oligonucleotide (ASO), and an aptamer. In embodiments, the protein is an antibody.

In embodiments, the subject has undergone or will undergo a lung transplantation. In embodiments, the method further comprises performing a lung transplantation in the subject. In embodiments, the P2Y2R inhibitor is administered before the lung transplantation. In embodiments, the P2Y2R inhibitor is administered at the same time as the lung transplantation. In embodiments, the P2Y2R inhibitor is administered after the lung transplantation. In embodiments, the P2Y2R inhibitor is administered between about one hour before the lung transplantation and about one hour after the lung transplantation procedure. In embodiments, the P2Y2R inhibitor is administered intravenously.

In embodiments, the method further comprises administering to the subject at least one of a TRPV4 inhibitor and a Panx1 inhibitor. In embodiments, each of the at least one TRPV4 inhibitor and Panx1 inhibitor is selected from a compound, a protein, and an inhibitory nucleic acid. In embodiments, the TRPV4 inhibitor is GSK2193874. In embodiments, the Panx1 inhibitor is spironolactone. In embodiments, the TRPV4 inhibitor and the Panx1 inhibitor are administered at the same time as the P2Y2R inhibitor.

### BRIEF DESCRIPTION OF THE DRAWINGS

FIGS. 1A-1J. Lung I R increases TRPV4 channel activity in the pulmonary endothelium. (FIG. 1A) Left-lung hilar-ligation model of IRI (see Methods). Mice in the IR group underwent left lung hilar ligation for 1 hour followed by reperfusion for 2 hours. Control mice underwent sham surgery with no hilar ligation. (FIG. 1B) Left, representative images of left lungs from TRPV4fl/fl sham, TRPV4fl/fl IR, and TRPV4EC−/− IR mice showing gross injury after IR and protection in TRPV4EC−/− mice. Center, image showing the ordering system used for dissecting out small PAs (4th-order, dashed area) from left lungs for analysis of overall Ca²⁺ signals and TRPV4 sparklet activity. Right, grayscale image showing approximately 15 ECs from a field of view in a PA loaded with Fluo-4 AM. Dashed outline shows one EC. (FIG. 1C) Representative fractional fluorescence (F/F0) traces showing baseline Ca²⁺ signaling activity in the pulmonary endothelium from TRPV4fl/fl sham, TRPV4fl/fl IR, TRPV4EC−/− sham, and TRPV4EC−/− IR mice. (FIG. 1D) Upward deflection from the baseline represents a Ca²⁺ signal. Each trace indicates Ca²⁺ signals from a different site in a field of view. Several active sites can be found in each field of view. Total baseline Ca 2 signaling activity, represented as sites per cell (top) and events per cell (bottom), in PAs from TRPV4fl/fl sham, TRPV4fl/fl IR, TRPV4EC−/− sham, and TRPV4EC−/− IR mice, with and without the TRPV4 inhibitor GSK219 (100 nM) (n=5-6/group, *P<0.05, **P<0.01; ns, not significant; two-way ANOVA). (FIG. 1E) Representative F/F0 traces reflecting TRPV4 sparklet activity in the presence of cyclopiazonic acid (CPA, 20 μM), used to eliminate intracellular Ca²⁺ release signals, in the pulmonary endothelium from TRPV4fl/fl sham, TRPV4fl/fl IR, TRPV4EC−/− sham, and TRPV4EC−/− IR mice. Dotted lines represent quantal levels (single-channel amplitudes) determined from all-point histograms. 11 (FIG. 1F) TRPV4 sparklet activity per site (left: NPO per site, where N is the number of channels and PO is the open-state probability) and TRPV4 sparklet sites per cell (right) in the presence of CPA in the pulmonary endothelium from TRPV4fl/fl sham, TRPV4fl/fl IR, TRPV4EC−/− sham, and TRPV4EC−/− IR mice (n=5-6/group; *P<0.05, ***P<0.001; ns, not significant; two-way ANOVA). (FIG. 1G) TRPV4 mRNA levels in freshly isolated pulmonary endothelium from TRPV4fI/fl mice, expressed relative to sham (n=4) and IR (n=5) (ns, not significant; unpaired t-test). (FIG. 1H) Baseline endothelial Ca²⁺ signaling activity in small PAs from TRPV4fl/fl mice in the presence of apyrase (10 U/mL), an eATP-diphosphohydrolase, presented as sites per cell (left) and events per cell (right) (n=5-6/group; ***P<0.001; two-way ANOVA). (FIG. 1I) Endothelial TRPV4 sparklet sites per cell and sparklet activity in small PAs from TRPV4fl/fl mice in the presence of CPA in pulmonary endothelium from TRPV4fl/fl sham, TRPV4fl/fl IR, and TRPV4EC−/− IR mice (n=5-6/group; ***P<0.001; two-way ANOVA). (FIG. 1J) Diagram depicting induction of TRPV4 Ca²⁺ channel activity by eATP after lung IR, leading to elevated intracellular Ca²′.

FIGS. 2A-2I. Lung I R increases TRPV4 sparklet activity via P2Y2R signaling in the pulmonary endothelium. Ca²⁺ signals were recorded from the endothelium of Fluo-4-loaded small PAs. (FIG. 2A) Total Ca²⁺ signaling activity (sites per cell and events per cell) and (FIG. 2B) TRPV4 sparklet activity (sparklet sites per cell and sparklet activity per site) in ECs from small PAs of C57BL6/J mice in the absence and presence of the P2Y2R inhibitor, AR-C 118925XX (ARC, 10 μM) (n=5-6/group: ***P<0.001; ns, not significant; two-way ANOVA). (FIG. 2C) Representative images of left lungs from P2Y2Rfl/fl sham, P2Y2Rfl/fl IR, and P2Y2REC−/− IR mice showing gross lung injury after IR and protection in P2Y2REC-/-mice. (FIG. 2D) Representative F/F0 traces showing baseline Ca²⁺ signaling activity in the pulmonary endothelium. (FIG. 2E) Dot plot showing total baseline Ca²⁺ signaling activity for P2Y2Rfl/fl sham, P2Y2Rfl/fl IR, P2Y2REC−/− sham, and P2Y2REC−/− IR mice, with and without the TRPV4 inhibitor GSK219 (100 nM) (n=5-6/group; **P<0.01, ***P<0.001; ns, not significant: two-way ANOVA). (FIG. 2F) Representative TRPV4 sparklet traces and (FIG. 2G) analysis of sparklet activity in the pulmonary endothelium from P2Y2Rfl/fl sham, P2Y2Rfl/fl IR, P2Y2REC−/− sham. and P2Y2REC−/− IR mice (n=5-6/group; **P<0.01, ***P<0.001; ns, not significant; two-way ANOVA). (FIG. 2H) TRPV4 mRNA levels in freshly isolated pulmonary ECs from P2Y2Rfl/fl mice (sham, n=4; IR, n=5: unpaired t-test). Data are expressed relative to the sham group. (FIG. 2I) Diagram depicting eATP-induced activation of endothelial P2Y2R signaling and Ca²⁺ influx through TRPV4 channels after lung IR.

FIGS. 3A-3E. Endothelium-specific P2Y2R deletion decreases IR-induced neutrophil infiltration and lung edema and improves lung function after IR. (FIG. 3A) Partial pressure of arterial oxygen (PaO₂), (FIG. 38) lung compliance, and (FIG. 3C) lung edema (wet/dry weight ratio), measured in P2Y2Rfl/fl sham, P2Y2Rfl/fl IR, P2Y2REC−/− sham, and P2Y2REC−/− IR mice (n=5-6/group: ***P<0.001; ns, not significant; two-way ANOVA). (FIG. 3D) Representative immunostaining images showing neutrophil infiltration (stained pink with alkaline phosphatase). Scale bar: 50 μm. Quantification of the number of neutrophils per high-powered field (HPF: right) from P2Y2Rfl/fl sham, P2Y2Rfl/fl IR, P2Y2REC−/− sham, and P2Y2REC−/− IR mice (n=5-6/group; ***P<0.001; two-way ANOVA). (FIG. 3E) Concentrations of the proinflammatory cytokines CXCL1/KC, CXCL2/MCP-1. IL-6, and TNFα in left-lung BAL fluid from P2Y2Rfl/fl sham. P2Y2Rfl/fl IR. P2Y2REC−/− sham, and P2Y2REC−/− IR mice (n=5-6/group; ***P<0.001; ns, not significant; two-way ANOVA).

FIGS. 4A-4E. Exposure of small PAs to acute HR induces endothelial TRPV4 sparklet activity through P2Y2R stimulation. En face small PAs were exposed to 1 hour hypoxia followed by 10 minutes reoxygenation (HR) or normoxia (N) for 70 minutes. The final 10 minutes included incubation with the Ca²⁺ indicator, Fluo-4 (10 PM), for each group. (FIG. 4A) HR model and (FIG. 48) analysis of baseline Ca²⁺ signaling activity and (FIG. 4C) TRPV4 sparklet activity after normoxia or HR in the pulmonary endothelium of small PAs from TRPV4fl/fl and TRPV4EC−/− mice, in the presence or absence of the TRPV4 inhibitor, GSK219 (100 nM) (n=5-6/group: ***P<0.001; ns, not significant; two-way ANOVA). (FIG. 4D) Total baseline Ca²⁺ signaling activity and (FIG. 4E) TRPV4 sparklet activity after normoxia or HR in the pulmonary endothelium of small PAs from P2Y2Rfl/fl and P2Y2REC-/-IR mice, in the presence and absence of the TRPV4 inhibitor GSK219 (n=5-6/group: *P<0.05. ***P<0.001; ns, not significant; two-way ANOVA).

FIGS. 5A-5G. Exposure to acute HR increases TRPV4 channel activity in human PMVECs. (FIG. 5A) Top, representative whole-cell patch-clamp traces of TRPV4 currents, defined as currents in the presence of the TRPV4 agonist GSK101 (10 nM) minus those in the presence of GSK101+the TRPV4 antagonist GSK219 (100 nM). in human PMVECs. Outward currents through TRPV4 channels were recorded using −100 mV to +100 mV voltage ramps. Experiments were performed in the presence of ruthenium red (RuR; 1 μM) to prevent Ca²⁺ influx through TRPV4 channels and subsequent Ca²⁺-dependent activation of K+ channels. Bottom, averaged outward currents in human PMVECs at +100 mV under basal conditions, in the presence of GSK101 (10 nM) or GSK101+GSK219 (100 nM), and GSK219-sensitive currents (GSK101 minus [GSK101+GSK219]) (*P<0.05, **P<0.01, ***P<0.001 vs. Basal; two-way ANOVA). (FIG. 5B) Calbryte 520-loaded human PMVECs and (FIG. 5C) representative traces and analyzed data showing total baseline Ca²⁺ signaling activity in the absence and presence of ARC (10 μM) in human PMVECs exposed to normoxia or HR. (FIG. 5D) Representative traces and (FIG. 5E) analyzed data showing TRPV4 sparklet activity in the absence and presence of ARC (10 M) in human PMVECs (n=5-7/group, ***P<0.001: two-way ANOVA). (FIG. 5F) Top, greyscale images showing ECs from fluo-4-loaded, freshly isolated small PAs from human lungs. Small P As were exposed to normoxia or HR. Bottom, CD31 immunostaining showing ECs in small PAs isolated from human lungs (left) and F/F0 traces showing baseline endothelial Ca²⁺ signaling activity in small PAs from human lungs (right). (FIG. 5G) Analyzed data showing total baseline endothelial Ca²⁺ signaling activity in the absence and presence of ARC in small PAs from human lungs.

FIGS. 6A-6G. Panx1-effluxed eATP mediates IR-induced activation of endothelial P2Y2R-TRPV4 signaling. (FIG. 6A) Representative images of left lungs from Panx1fl/fl sham, Panx1fl/fl IR, and Panx1EC−/− IR mice showing gross injury after IR and protection in Panx1EC−/− mice. (FIG. 6B) Bioluminescence measurements of eATP (nM) released from small PAs of Panx1fl/fl sham, Panx1fl/fl IR, PanxIEC−/− sham, and PanxIEC−/− IR mice (n=6/group; ***P<0.001: ns, not significant; two-way ANOVA). (FIG. 6C) Panx1 mRNA levels in freshly isolated pulmonary ECs from Panx1fl/fl sham (n=4) and Panx1ECfl/fl IR (n=5) mice (ns. not significant: unpaired t-test). Data are expressed relative to the sham group. (FIG. 6D) Total baseline Ca²⁺ signaling activity and (FIG. 6E) TRPV4 sparklet activity in the pulmonary endothelium from Panx1fl/fl sham, Panx1fl/fl IR, Panx1EC/IR, and Panx1EC−/− IR mice in the absence and presence of the TRPV4 inhibitor GSK219 (100 nM) (n=5-6/group, *P<0.05, **P<0.01. ***P<0.001; ns, not significant; two-way ANOVA). (FIG. 6F) Total baseline Ca²⁺ signaling activity and (FIG. 6G) TRPV4 sparklet activity in the pulmonary endothelium from Panx1fl/fl sham and Panx1EC−/− IR mice in the absence and presence of GSK219 (100 nM) (n=5-6/group, *P<0.05, *P<0.01, ***P<0.001; ns, not significant; two-way ANOVA).

FIG. 7. Outline of lung IR-induced Panx1-eATP-P2Y2R-TRPV4 signaling in pulmonary ECs.

FIGS. 8A-8B. Ischemia alone does not increase endothelial Ca²⁺ signaling activity in small PAs. (FIG. 8A) F/F0 traces showing baseline Ca²⁺ signaling activity from small PAs exposed to sham surgery or 1 hour of ischemia (no reperfusion). (FIG. 8B) Total endothelial Ca²⁺ signaling activity represented as sites per cell and events per cell in small PAs (n=3/group; ns, not significant; t-test).

FIGS. 9A-9B. Exposure of mouse PMVECs to acute HR induces endothelial TRPV4 channel activity. Mouse PMVECs were exposed to 1 h hypoxia followed by 10 minutes reoxygenation (HR) or normoxia (N) for 70 minutes. (FIG. 9A) GSK2193874 (GSK219, TRPV4 inhibitor, 100 nM)-sensitive currents through TRPV4 channels in mouse PMVECs under normoxia and HR conditions recorded in the presence of RuR (1 μM, left) and dot plot showing outward currents at +100 mV (right). (FIG. 9B) TRPV4 sparklet activity (sites per cell, left; activity per site, fight) after normoxia or HR exposure of mouse PMVECs in the absence and presence of GSK219 (n=5-6/group; *P<0.05: **P<0.01; ns, not significant; two-way ANOVA).

FIGS. 10A-10B. Hypoxia alone does not increase endothelial Ca²⁺ signaling activity in small PAs. (FIG. 10A) F/F0 traces showing baseline Ca²⁺ signaling activity in small PAs exposed to normoxia or chemically induced hypoxia (sodium dithionite, 1 mM). (FIG. 10B) Total baseline endothelial Ca²⁺ signaling activity represented as sites per cell and events per cell (n=3/group; ns. not significant; t-test).

FIG. 11. Activation of TRPV4 channels with synthetic agonist GSK01 increases eATP release from small PAs (A) eATP release (nM) from small PAs before and after addition of TRPV4 agonist GSK101 (10 nM) (n=10-14/group; **P<0.001; paired t-test).

FIGS. 12A-12B. A left-lung hilar-ligation, involving 1 hour of ligation (ischemia) followed by 2 hours of reperfusion was used. Partial pressure of arterial oxygen (PaO₂. A) and lung edema (wet/dry weight ratio. B) in sham and IR mice treated with vehicle (DMSO) or P2Y2R inhibitor AR-C 118925XX (AR-C, 5 mg/kg, when, what route) (n=6 mice, one-way ANOVA).

FIGS. 13A-13B. Studies were performed in murine PMVECs exposed to hypoxia-reoxygenation (6 and 4 hrs. respectively) or normoxia (10 hrs). PMVECs were treated with vehicle or P2Y2R inhibitor AR-C118925XX (AR-C, 5 μM). Concentrations of proinflammatory cytokines, KC and MCP-1, released from PMVECs in the culture media were measured (n=6-8, one-way ANOVA).

FIGS. 14A-14B. Lung function is improved during reperfusion in recipient animals treated with GSK219. FIG. 14A. Left lung-specific oxygenation (pulmonary vein PaO₂/FiO₂ ratio) was significantly improved in the GSK219-treated recipient animals vs. vehicle controls after 3 and 4 hours of reperfusion. *P=0.026, **P=0.003. FIG. 14B. Dynamic lung compliance was similar between groups. Measurements are at 15 min and then hourly during reperfusion. N=5/group. Data are expressed as mean±standard deviation.

FIG. 15 Lung wet/dry weight ratios after transplant and reperfusion. No significant differences in wet/dry weight ratios were observed between groups in either left upper lobe. left lingula, or left lower lobe. Data are presented using box-and-whisker plots where the lower and upper borders of the box represent the lower and upper quartiles (25th percentile and 75th percentile): the middle horizontal line represents the median; and the lower and upper whiskers represent the minimum and maximum data values. ns=not significant.

FIGS. 16A-16B. Histologic lung injury severity scores were improved in recipient animals treated with GSK219. FIG. 16A Histologic scores for alveolar edema and polymorphonuclear neutrophils per high-powered field (PMNs/HPF), as well as composite score, were significantly improved after reperfusion in GSK219-treated animals versus vehicle. Data are presented using box-and-whisker plots where the lower and upper borders of the box represent the lower and upper quartiles (25th percentile and 75th percentile); the middle horizontal line represents the median; and the lower and upper whiskers represent the minimum and maximum data values. *P=0.048,**P=0.028, ***P=0.041, ns=not significant. FIG. 16B. Representative H&E-stained lung sections after 4 hours of reperfusion in vehicle- and GSK219-treated recipient animals (20× magnification).

FIGS. 17A-17B. Treatment of recipient animals with GSK219 attenuated neutrophil infiltration in transplanted lungs. FIG. 17A. Neutrophil numbers in bronchoalveolar lavage (BAL) fluid measured by differential cell count (% total count) were significantly reduced by GSK219 treatment versus vehicle. FIG. 17B. The number of neutrophils per high-powered field (HPF), as measured by immunohistochemical staining of lung sections, were significantly reduced by GSK219 treatment versus vehicle. Quantification is shown on the left, and representative images (20× magnification) are shown on the right where neutrophils are stained red. Data are presented using box-and-whisker plots where the lower and upper borders of the box represent the lower and upper quartiles (25th percentile and 75th percentile); the middle horizontal line represents the median; and the lower and upper whiskers represent the minimum and maximum data values. *P=0.004, **P<0.001.

FIGS. 18A-18C. Treatment of recipient animals with GSK219 attenuated expression of endothelial cell adhesion molecules in lungs after reperfusion. Expression of P-selectin in BAL fluid was significantly reduced in GSK219-treated animals versus vehicle FIG. 18A. No significant changes were detected in expression of VCAM1 or ICAM-1 (FIGS. 18B and 18C), which trended lower in GSK219- treated animals. Data are presented using box-and-whisker plots where the lower and upper borders of the box represent the lower and upper quartiles (25th percentile and 75th percentile); the middle horizontal line represents the median; and the lower and upper whiskers represent the minimum and maximum data values. *P=0.0075.

FIGS. 19A-19D. Treatment of recipient animals with GSK219 reduced tight junction protein levels in BAL fluid after reperfusion. Concentrations of tight junction proteins occludin (FIG. 19A), claudin-5 (FIG. 19B), and ZO-1 (FIG. 19C) in bronchoalveolar lavage (BAL) fluid were significantly reduced in recipient animals treated with GSK219 versus vehicle. The expression of ZO-2 in BAL fluid (FIG. 19D) was not significantly different but trended lower. Data are presented using box-and-whisker plots where the lower and upper borders of the box represent the lower and upper quartiles (25th percentile and 75th percentile); the middle horizontal line represents the median; and the lower and upper whiskers represent the minimum and maximum data values. *P<0.05.

FIGS. 20A-20D. Treatment of recipient animals with GSK219 reduced expression of inflammatory markers after reperfusion. Expression of HMGB1 in bronchoalveolar lavage (BAL) fluid (FIG. 20A) and Ang-2 in plasma (FIG. 20B) were significantly reduced in animals treated with GSK219 versus vehicle. Expression of IL-8 (FIG. 20C), but not IL-1 (FIG. 20D), was significantly reduced in BAL fluid of GSK219-treated animals. Data are presented using box-and-whisker plots where the lower and upper borders of the box represent the lower and upper quartiles (25th percentile and 75th percentile); the middle horizontal line represents the median; and the lower and upper whiskers represent the minimum and maximum data values. *P<0.03, **P=0.001.

FIG. 21. Systemic arterial oxygenation during reperfusion. Systemic arterial oxygenation (PaO₂/FiO₂ ratio) improved during reperfusion in GSK-treated recipients compared to vehicle, although this was not significant. N=5/group. Data are expressed as mean±standard deviation.

FIGS. 22A-22C. Treatment of recipient animals with GSK219 did not alter plasma levels of cell adhesion molecules after reperfusion. Concentrations of P-selectin (FIG. 22A), VCAM-1 (FIG. 22B), and ICAM-1 (FIG. 22C) in plasma after reperfusion were not significantly different between GSK219- and vehicle-treated animals. Data are presented using box-and-whisker plots where the lower and upper borders of the box represent the lower and upper quartiles (25th percentile and 75th percentile); the middle horizontal line represents the median; and the lower and upper whiskers represent the minimum and maximum data values.

FIGS. 23A-23D. Claudin-5 and ZO-1 expression in plasma was reduced by GSK219 treatment of recipient animals. After reperfusion, expression of claudin-5 (FIG. 23B) and ZO-1 (FIG. 23C), but not occludin (FIG. 23A) or ZO-2 (FIG. 23D) were significantly reduced in plasma of recipient animals treated with GSK219 versus vehicle. Data are presented using box-and-whisker plots where the lower and upper borders of the box represent the lower and upper quartiles (25th percentile and 75th percentile); the middle horizontal line represents the median; and the lower and upper whiskers represent the minimum and maximum data values. *P=0.014, **P=0.035.

### DETAILED DESCRIPTION OF THE INVENTION

Lung ischemia-reperfusion injury (IRI), characterized by robust inflammation, increased vascular permeability, and lung edema, is the major cause of primary graft dysfunction after lung transplantation, and results after lung ischemia-reperfusion (IR). The inventors recently reported that endothelial cell (EC) transient receptor potential vanilloid 4 (TRPV4EC) channels play a crucial role in lung edema and dysfunction after IR. The levels of extracellular adenosine triphosphate (eATP), which can activate TRPV4Ec channels through purinergic P2Y2 receptors (P2Y2Rs), are elevated during lung IRI. As shown in the Examples, the inventors found that disrupting the endothelial Panx1-P2Y2R-TRPV4 signaling axis by inhibiting or deleting the P2Y2R reduces lung edema, inflammation, and dysfunction after IR.

Accordingly, provided herein is a method for preventing or treating lung ischemia-reperfusion injury (IRI) in a subject in need thereof, the method comprising administering to the subject a P2Y2 receptor (P2Y2R) inhibitor.

The terms “treating,” “treat” and “treatment” include (i) preventing a disease. pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate. Treating I R includes reducing, repressing, delaying or preventing lung IR.

The term “subject” may be used interchangeably with the terms “individual” and “patient” and includes human and non-human mammalian subjects. As used herein, “a subject in need thereof” refers to a subject having or at risk of developing lung IR or symptoms thereof.

An “inhibitor” of P2Y2R is an agent that reduces the function of P2Y2R. The inhibitor may inhibit or reduce the expression or activity of P2Y2R. The inhibitor may be one or more of a compound, a protein, and an inhibitory nucleic acid.

The P2Y2R inhibitor may be a chemical compound. The inhibitor may comprise AR-C 118925XX (ARC) or a pharmaceutically acceptable salt thereof. A “compound,” as used herein, refers to any type of substance or agent that is commonly considered a drug. or a candidate for use as a drug, as well as combinations and mixtures of the above. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by one of skill in the art that most or all of the compounds as disclosed herein are capable of forming salts and that the salt forms of pharmaceuticals are commonly used. often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid. succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate. acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate. mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein may not be critical to the activity of the compound. so long as the salt as a whole is pharmacologically acceptable and as long as the counter-ion does not contribute undesired qualities to the salt as a whole. Undesired qualities may include undesirable solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The P2Y2R inhibitor may be prepared as a formulation or pharmaceutical composition. Inert ingredients and manner of formulation of the pharmaceutical compositions are conventional. The pharmaceutical compositions may be formulated according to conventional pharmaceutical practice (see, e.g., Remington: The Science and Practice of Pharmacy. 20th edition, 2000. ed. A. R. Gennaro, Lippincott Williams & Wilkins, Philadelphia. and Encyclopedia of Pharmaceutical Technology, eds. J. Swarbrick and J. C. Boylan, 1988-1999, Marcel Dekker, New York). The pharmaceutical compositions may be designed or intended for oral, rectal, nasal, systemic, topical or transmucosal (including buccal, sublingual, ocular, vaginal and rectal) and parenteral (including subcutaneous, intramuscular, intravenous, intraarterial, intradermal, intraperitoneal, intrathecal, intraocular and epidural) administration. Aqueous and non-aqueous liquid or cream formulations may be delivered by a parenteral, oral or topical route. The compositions may be present as an aqueous or a non-aqueous liquid formulation or a solid formulation suitable for administration by any route, e.g., oral, topical, buccal. sublingual, parenteral. aerosol, a depot such as a subcutaneous depot or an intraperitoneal or intramuscular depot. The pharmaceutical compositions may be lyophilized. The pharmaceutical compositions may contain auxiliary substances such as wetting or emulsifying agents, pH buffering agents. gelling or viscosity enhancing additives, preservatives, flavoring agents, colors, and the like, depending upon the route of administration and the preparation desired.

In preferred embodiments, the P2Y2R inhibitor is administered intravenously. For intravenous administration, suitable carriers include physiological saline. bacteriostatic water, Cremophor E L® (BASF, Parsippany, N.J., USA) or phosphate buffered saline (PBS). In all cases, a composition for parenteral administration must be sterile and should be formulated for ease of injectability. The composition should be stable under the conditions of manufacture and storage, and must be shielded from contamination by microorganisms such as bacteria and fungi.

The composition may further comprise a pharmaceutically acceptable carrier. The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, com starch and potato starch; cellulose and its derivatives such as, but not limited to, sodium carboxymethyl cellulose, ethyl cellulose and cellulose acetate; powdered tragacanth; malt: gelatin; talc; excipients such as, but not limited to, cocoa butter and suppository waxes; oils such as, but not limited to, peanut oil, cottonseed oil, safflower oil, sesame oil, olive oil, com oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water: isotonic saline: Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutically acceptable carriers are well known to those skilled in the art and include, but are not limited to, 0.01 to 0.1 M and preferably 0.05M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil. com oil and soybean oil: glycols, such as propylene glycol: polyols such as glycerin. sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid. ethylenediamine tetraacetic acid (EDTA), sorbitol, tartaric acid, phosphoric acid and the like.

The composition may additionally include a biologically acceptable buffer to maintain a pH close to neutral (7.0-7.3). Such buffers preferably used are typically phosphates, carboxylates. and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final composition. The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems. the terms “about” and “approximately” may mean values that are within an order of magnitude. preferably within 5-fold and more preferably within 2-fold of a given value. Numerical quantities given herein are approximate unless stated otherwise, meaning that the term “about” or “approximately” can be inferred when not expressly stated.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician. the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders. Tablets are prepared by direct compression, by wet granulation, or by dry granulation. Their formulations usually incorporate diluents, binders. lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects. A lubricant can be used in a tablet formulation to prevent the tablet and punches from sticking in the die. The lubricant can be chosen from such slippery solids as talc, magnesium and calcium stearate, stearic acid, and hydrogenated vegetable oils. Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate. hydroxypropyl methylcellulose phthalate. and hydroxypropyl methylcellulose acetate succinate.

Transdermal patches can also be used to deliver the compositions. Transdermal patches can include a resinous composition in which a compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The P2Y2R inhibitor may be a protein. As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids. A protein typically comprises a polymer of naturally or non-naturally occurring amino acids (e.g., alanine, arginine, asparagine, aspartic acid, cysteine, glutamine, glutamic acid, glycine, histidine. isoleucine. leucine, lysine, methionine, phenylalanine, proline. serine. threonine. tryptophan, tyrosine, and valine).

The term “amino acid” is used interchangeably with “amino acid residue,” and may refer to a free amino acid and to an amino acid residue of a peptide/protein. It will be apparent from the context in which the term is used whether it refers to a free amino acid or a residue of a peptide/protein.

The expression “amino acid” as used herein is meant to include both natural and synthetic amino acids, and both D and L amino acids. “Standard amino acid” means any of the twenty standard L-amino acids commonly found in naturally occurring peptides/proteins. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides/proteins of the present invention, and particularly at the carboxy- or amino-terminus, can be modified by methylation, amidation, acetylation or substitution with other chemical groups which can change the peptide's/protein's circulating half-life without adversely affecting their activity (e.g., peptidomimetic for making peptides protease resistant).

Proteins described herein include antibodies. The term “antibody” as used herein refers to immunoglobulin molecules or other molecules which comprise an antigen binding domain. The term “antibody” is thus intended to include whole antibodies (e.g., IgG, IgA, IgE, IgM, or IgD), monoclonal antibodies, chimeric antibodies, humanized antibodies, and antibody fragments, including single chain variable fragments (ScFv), single domain antibodies (nanobodies), and antigen-binding fragments, genetically engineered antibodies, among others, as long as the characteristic properties (e.g., ability to bind CD30) are retained. The term “antibody fragment” as used herein is intended to include any appropriate antibody fragment that displays antigen binding function, for example, Fab, Fab′, F(ab′)2, scFv, Fv, dsFv, ds-scFv, Fd, mini bodies, monobodies, and multimers thereof and bispecific antibody fragments.

Antibody fragments/antigen binding fragments comprise an antigen binding domain. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (e.g. single chain antibodies or single chain Fv (scFv), (see for instance Bird et al. , Science 242, 423-426 (1988) and Huston et al., PNAS USA 85, 5879-5883 (1988)). The term “fragment” as used herein refers to fragments of biological relevance (functional fragment), e.g., fragments which can contribute to or enable antigen binding, e.g., form part or all of the antigen binding site or can contribute to the prevention of the antigen interacting with its natural ligands. Fragments may comprise a heavy chain variable region (V_{H} domain) and light chain variable region (V_{L}). Fragments may comprise one or more of the heavy chain complementarity determining regions (CDRHs) of the antibodies or of the V_{H} domains, and one or more of the light chain complementarity determining regions (CDRLs), or V_{L} domains to form the antigen binding site.

Antibodies can be genetically engineered from CDRs and monoclonal antibody sequences by using conventional techniques such as. for example. synthesis by recombinant techniques or chemical synthesis. Techniques for producing antibody fragments are well known and described in the art. One may wish to engraft one or more CDRs from monoclonal antibodies into alternate scaffolds. For example, standard molecular biological techniques can be used to transfer the DNA sequences encoding the antibody's CDR(s) to (1) full IgG scaffold of human or other species; (2) a scFv scaffold of human or other species, or (3) other specialty vectors. If the CDR(s) have been transferred to anew scaffold all of the previous modifications described can also be performed. For example, one could consult Biotechnol Genet Eng Rev, 2013, 29:175-86 for a review of useful methods.

The antibodies can be wholly or partially synthetically produced. Thus, the antibody may be from any appropriate source, for example recombinant sources and/or produced in transgenic animals or transgenic plants. Thus, the antibody can be produced in vitro or in vivo. The antibody may comprise all or a portion of a heavy chain constant region, such as an IgG1, IgG2, IgG3, IgG4, IgAQ1, IgA2, IgE. IgM or IgD constant region. The antibody may comprise all or a portion of a kappa light chain constant region or a lambda light chain constant region. All or part of such constant regions may be produced wholly or partially synthetic. Appropriate sequences for such constant regions are well known and documented in the art.

The term “complementarity determining regions” or “CDRs,” as used herein, refers to part of the variable chains in immunoglobulins (antibodies) and T cell receptors, generated by B-cells and T-cells respectively, where these molecules bind to their specific antigen. As the most variable parts of the molecules, CDRs are crucial to the diversity of antigen specificities generated by lymphocytes. There are three CDRs (CDR1, CDR2 and CDR3), arranged non-consecutively, on the amino acid sequence of a variable domain of an antigen binding site.

Since the antigen binding sites are typically composed of two variable domains (on two different polypeptide chains, heavy and light chain), there are six CDRs for each antigen binding site that can collectively come into contact with the antigen. A single whole antibody has two antigen binding sites and therefore contains twelve CDRs. Sixty C DRs can be found on a pentameric IgM molecule.

Within the variable domain, CDR1 and CDR2 may be found in the variable (V) region of a polypeptide chain, and CDR3 includes some of V, and all of diversity (D, heavy chains only) and joining (J) regions. Since most sequence variation associated with immunoglobulins and T cell receptors is found in the CDRs, these regions are sometimes referred to as hypervariable regions. Among these, CDR3 shows the greatest variability as it is encoded by a recombination of VJ in the case of a light chain region and VDJ in the case of heavy chain regions. The tertiary structure of an antibody is important to analyze and design new antibodies. The human V_{H} complex is composed of approximately 100 gene segments per haploid genome, including at least 51 functional genes, as judged by successful rearrangement in cloned cDNA. On the basis of nucleic acid sequence homology, the V_{H} genes have been grouped into 6-7 families (VH 1-7). Among the seven families, the VH3 family is the largest. The antibody disclosed herein may be derived from the VH3-74 family or the VH3-33 family or its paralog.

The terms “monoclonal antibody” or “monoclonal antibody composition” as used herein refer to a preparation of an antibody of a single amino acid composition that specifically binds to a single epitope of the antigen.

The term “chimeric antibody” refers to an antibody comprising a variable region, i.e., binding region, from one source or species and at least a portion of a constant region derived from a different source or species, usually prepared by recombinant DNA techniques. Other forms of “chimeric antibodies” are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. The antibodies may be chimeric antibodies including heavy chain constant domains from non-human mammals (e.g., mouse, rat, rabbit, or non-human primate). The antibodies may be chimeric antibodies including constant regions from rabbit heavy chain immunoglobulin sequences. Suitable heavy chain constant region sequences from non-human mammals, including mouse, rat, rabbit, and non-human primate are known in the art.

The antibody may be a human antibody, as it may include the constant region from human germline immunoglobulin sequences. The term “recombinant human antibody” includes all human antibodies that are prepared, expressed, created, or isolated by recombinant means, such as antibodies isolated from a host cell such as an SP2-0, NSO or CHO cell (like CHO Kl) or from an animal (e.g., a mouse) that is transgenic for human immunoglobulin genes or antibodies expressed using a recombinant expression vector transfected into a host cell. Such recombinant human antibodies have variable and, in some embodiments, constant regions derived from human germline immunoglobulin sequences in a rearranged form.

The P2Y2R inhibitor may be an inhibitory nucleic acid. An inhibitory nucleic acid may reduce the expression of the P2Y2R by binding to the coding region or regulatory regions of the gene or mRNA encoding P2Y2R and preventing translation of the P2Y2R protein. Inhibitory nucleic acids include, but are not limited to, double stranded RNA (dsRNA), small interfering RNA (siRNA), short hairpin RNA (shRNA), microRNA (miRNA), artificial microRNA (amiRNA): antisense oligonucleotide (ASO), and nucleic acid aptamers.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

Short twenty-one to twenty-five nucleotide double-stranded RNAs are effective at down-regulating gene expression (Zamore et al., Cell 101: 25-33: Elbashir et al., Nature 411: 494-498, 2001, hereby incorporated by reference). The therapeutic effectiveness of an sirNA approach in mammals was demonstrated in vivo by McCaffrey et al. (Nature 418: 38-39.2002). Given the sequence of a target gene, siRNAs may be designed to inactivate that gene. Such siRNAs, for example, could be administered directly to an affected tissue, or administered systemically. The nucleic acid sequence of an Parl gene can be used to design small interfering RNAs (siRNAs). The 21 to 25 nucleotide siRNAs may be used, for example, as therapeutics to treat lupus.

A double-stranded RNA (dsRNA) molecule may include between eight and nineteen consecutive nucleobases of a nucleobase oligomer of the disclosure. The dsRNA can be two distinct strands of RNA that have duplexed, or a single RNA strand that has self-duplexed (small hairpin (sh)RNA). Typically. dsRNAs are about 21 or 22 base pairs. but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

A microRNA (miRNA) may include any type of interfering RNAs, including but not limited to, endogenous microRNAs and artificial microRNAs (amiRNA) (e.g., synthetic miRNAs). Endogenous microRNAs are small RNAs naturally encoded in the genome which are capable of modulating the productive utilization of mRNA. An artificial microRNA can be any type of RNA sequence, other than endogenous microRNA, which is capable of modulating the activity of an mRNA. A microRNA sequence can be an RNA molecule composed of any one or more of these sequences. MicroRNA sequences have been described in publications such as, Lim, et al., 2003, Genes & Development, 17, 991-1008, Lim et al., 2003, Science, 299, 1540, Lee and Ambrose, 2001, Science, 294, 862. Lau et al., 2001, Science 294, 858-861, Lagos-Quintana et al., 2002, Current Biology, 12, 735-739, Lagos-Quintana et al., 2001, Science, 294, 853-857, and Lagos-Quintana et al., 2003, RNA, 9, 175-179, which are incorporated herein by reference. Examples of microRNAs include any RNA that is a fragment of a larger RNA or is a miRNA, siRNA, stRNA, sncRNA, tncRNA, snoRNA, smRNA, snRNA, or other small non-coding RNA. See. e.g., US Patent Applications 20050272923, 20050266552, 20050142581, and 20050075492. A microRNA precursor (or pre-miRNA) refers to a nucleic acid having a stem-loop structure with a microRNA sequence incorporated therein. A mature microRNA includes a microRNA that has been cleaved from a pre-miRNA, or that has been synthesized (e.g., synthesized in a laboratory by cell-free synthesis), and has a length of from about 19 nucleotides to about 27 nucleotides, e.g., a mature microRNA can have a length of 19 nt, 20 nt, 21 nt, 22 nt, 23 nt, 24 nt, 25 nt, 26 nt, or 27 nt. A mature microRNA can bind to a target mRNA and inhibit translation of the target mRNA.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby. when the first and second portions are arranged in an antiparallel fashion, at least about 50%, including at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

The terms “nucleic acid” and “nucleic acid molecule,” as used herein, refer to a compound comprising a nucleobase and an acidic moiety. e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules. in which adjacent nucleotides are linked to each other via a phosphodiester linkage. As used herein, the terms “oligonucleotide” and “polynucleotide” can be used interchangeably to refer to a polymer of nucleotides (e.g., a string of at least three nucleotides). Nucleic acids encompass RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine): nucleoside analogs (e.g., 2-aminoadenosine. 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, 0(6)-methylguanine, and 2-thiocytidine); chemically modified bases: biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

“Encoding” refers to the inherent property of specific sequences of nucleotides in a polynucleotide, such as a gene, a cDNA, or an mRNA. to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions.” Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991. Critical Review in Biochem. and Mol. Biol. 26(3/4):227-259; and Owczarzy et al., 2008, Biochemistry, 47: 5336-5353, which are incorporated herein by reference).

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, poly amine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

As used herein, the term “administering”, refers to dispensing, delivering, or applying the P2Y2R inhibitor, to a subject by any suitable route for delivery of the inhibitor to the desired location in the subject, including delivery by either the parenteral or oral route, intramuscular injection, subcutaneous/intradermal injection, intravenous injection, intraperitoneal injection, intrathecal administration, buccal administration, transdermal delivery, topical administration, and administration by the intranasal or respiratory tract route.

The method may comprise administering a therapeutically effective amount of the P2Y2R inhibitor to the subject. The terms “effective amount” or “therapeutically effective amount” refer to an amount sufficient to effect beneficial or desirable biological and/or clinical results. The amount of the agent or pharmaceutical composition that is therapeutically effective may vary depending on the particular condition of the subject. Appropriate dosages may be determined, for example, by extrapolation from cell culture assays, animal studies, or human clinical trials taking into account body weight of the patient, absorption rate, half-life, disease severity and the like. The dosage lies preferably within a range of circulating concentrations that include the ED50 with little or no toxicity. The dosage may vary within this range depending upon the dosage form employed and the route of administration utilized. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In preferred embodiments, the P2Y2R inhibitor is administered one time. However, the P2Y2R inhibitor may be administered as a chronic treatment, meaning a treatment longer than one day or one dose. Compositions can be formulated in a unit dosage form, each dosage containing from about 1 to about 500 mg of each compound individually or in a single unit dosage form, such as from about 5 to about 300 mg, from about 10 to about 100 mg. and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

In embodiments, the subject has undergone or will undergo a lung transplantation. Further, the method may comprise performing a lung transplantation.

The P2Y2R inhibitor may be administered before the lung transplantation procedure beings, during the lung transplantation procedure, or after the lung transplantation procedure is completed. In preferred embodiments, the P2Y2R inhibitor is administered between about one hour before the lung transplantation procedure and about one hour after the lung transplantation procedure.

The method may further comprise administering at least one of a TRPV4 inhibitor and a pannexin 1 (Panx1) inhibitor. As shown in the Examples, in two separate models, lung IR increases endothelial Panx1 channel-mediated efflux of eATP, which then activates endothelial P2Y2R signaling, which then increases the activity of endothelial TRPV4 channels, resulting in endothelial barrier disruption, lung edema, immune cell infiltration, and lung dysfunction after IR. Targeting this signaling pathway helps alleviate IRI.

The TRPV4 and Panx1 inhibitors may be selected from a compound, a protein, and an inhibitory nucleic acid. In exemplary embodiments, the TRPV4 inhibitor is GSK2193874. The TRPV4 and/or Panx1 inhibitor may be administered at the same time as the P2Y2R inhibitor.

### Miscellaneous

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul. et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program. using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As will be understood by the skilled artisan, all numbers, including those expressing quantities of ingredients. properties such as molecular weight. reaction conditions, and so forth. are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

The term “standard,” refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges. are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly. provisos may apply to any of the disclosed categories or embodiments whereby any one or more of the recited elements, species. or embodiments, may be excluded from such categories or embodiments, for example, for use in an explicit negative limitation.

Methods involving conventional molecular biology techniques are described herein. Such techniques are generally known in the art and are described in detail in methodology treatises, such as Molecular Cloning: A Laboratory Manual. 2nd ed., vol. 1-3. ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers. Tetra. Letts. 22: 1859-1862, 1981, and Matteucci et al., J. Am. Chem. Soc. 103:3185, 1981.

### EXAMPLES

### Example 1

### Introduction

The success of lung transplantation is limited by a high rate of primary graft dysfunction (PGD) due to ischemia-reperfusion injury (IRI).^{1,2} PGD is the leading cause of death in the first 30 days following lung transplant and is also an independent risk factor for chronic allograft dysfunction.^{3,4} Lung IRI is a sterile inflammatory process characterized by robust oxidative stress, endothelial dysfunction, vascular permeability, edema, and alveolar damage after transplant.^{5,6} While a number of signaling pathways are involved in lung IRI, it is clear that the cellular release of adenosine triphosphate (ATP) plays a prominent role.^{1,5} Extracellular ATP (eATP) is an endogenous activator of purinergic receptor signaling. Therefore, targeting purinergic receptor-dependent mechanisms can be of therapeutic value in lung IRI. In the present study, we focused on the signaling mechanisms through which eATP and purinergic receptor signaling regulate vascular inflammation, edema, and dysfunction—the hallmarks of lung IRI.

Endothelial dysfunction and increased endothelial cell (EC) permeability are contributors to immune cell infiltration into pulmonary tissue and alveolar space, leading to lung edema after IR. EC pannexin 1 (Panx1) is a source of eATP in the intact pulmonary endothelium under normal conditions.^{7,8} We previously showed that endothelium-specific Panx1-knockout (PanxIEC−/−) mice are protected against vascular permeability, inflammation, and edema after lung IR,⁹ suggesting an important role for endothelial Panx1 and release of eATP in the pathogenesis of lung IRI. However, eATP-induced downstream signaling mechanisms that mediate lung IRI remain unknown.

Transient receptor potential vanilloid 4 (TRPV4) channels, which constitute a major Ca²⁺-entry pathway in pulmonary endothelium,^{8,10,11} have emerged as central players in the pathogenesis of different forms of lung injury.¹²⁻¹⁵ We provided the first evidence that endothelial TRPV4-knockout (TRPV4EC−/−) mice are protected against increased vascular permeability, lung inflammation, and edema after lung IR,¹⁶ suggesting that endothelial TRPV4 channels are mediators of lung IRI. However, whether increased activity of pulmonary endothelial TRPV4 channels contributes to lung IRI is not known. Recent studies provide evidence that, under healthy conditions, eATP activates endothelial TRPV4 channels through P2Y2R signaling.⁸ We demonstrated herein that eATP. effluxed via endothelial Panx1 channels, activates endothelial P2Y2R-TRPV4 channel signaling to mediate lung inflammation, edema, and dysfunction in lung IRI.

We investigated the role of endothelial Panx1-P2Y2R-TRPV4 signaling in lung IRI using tamoxifen-inducible, endothelium-specific P2Y2R,¹⁷ Panx1.¹⁸ and TRPV4¹⁹ knockout mice in combination with in vivo, ex vivo, and in vitro models of lung IRI. Our data show that eATP, derived by efflux via endothelial Panx1 channels, activates endothelial TRPV4 channels through P2Y2R signaling to promote lung inflammation and edema, and impair lung function in IRI. These data identify P2Y2R as a novel intermediate in an endothelial Panx1-P2Y2R-TRPV4 signaling axis and provide proof of principle that disrupting this pathway can be a potential treatment strategy for preventing IRI and PGD after lung transplantation.

### Materials and Methods

Animal Models and Human Tissue. All animal protocols were approved by the University of Virginia Animal Care and Use Committee. C57BL6/J and endothelium-specific P2Y2R. TRPV4, and Panx1 knockout mice were used. Isolation of small pulmonary arteries from human lungs was approved by University of Virginia Institutional Review Board (protocol 19044).

Murine lung IR model. A left-lung hilar-ligation model of IRI in mice involving 1 hour of ligation (ischemia) followed by 2 hours of reperfusion.^{9,20} Partial pressure of oxygen, lung compliance, lung edema, neutrophil infiltration, and cytokine levels in bronchoalveolar lavage fluid were assessed. Exposure of small pulmonary arteries and pulmonary microvascular endothelial cells to acute hypoxia-reoxygenation were used as ex vivo and in vitro surrogate models of IR, respectively.

Imaging endothelial Ca²⁻ signals. Ca²⁺ imaging studies were performed in mouse and human small pulmonary arteries and pulmonary microvascular endothelial cells with a spinning-disk confocal imaging system and electron-multiplying charge-coupled device camera.

Patch Clamp in ECs. Currents through TRPV4 channels were recorded in mouse and human PMVECs using whole-cell patch clamp.

Statistics. Data were analyzed with 2-tailed paired or independent t test, 1-way analysis of variance (with Tukey correction for multiple comparisons). or 2-way analysis of variance (with Bonferroni correction for multiple comparisons). Statistical significance was determined as a value of P<0.05.

Drugs and chemicals: Cyclopiazonic acid (CPA, depletes internal stores of Ca²⁺), GSK2193874 (GSK219. TRPV4 inhibitor). GSK1016790A (GSK101. TRPV4 agonist), AR-C 118925XX (ARC, P2Y2R inhibitor) and Ruthenium Red (TRPV4 channel blocker) were purchased from Tocris Bioscience (Minneapolis, MN). Fluo-4 AM (Ca²⁺ indicator) was purchased from Invitrogen (Carlsbad, CA). Tamoxifen and apyrase were obtained from Sigma-Aldrich (St. Louis, MO). Calbryte™ 520 AM (Ca²⁺ indicator) was purchased from AAT Bioquest (Pleasanton, CA). Pentobarbital was purchased from Sagent Pharmaceuticals (Schaumburg, IL). All other chemicals were purchased from Sigma-Aldrich.

Animals: All animal protocols were approved by the University of Virginia Animal Care and Use Committee. All experiments were performed in 10-14-week-old mice on a C57BL6/J background, in at least two independent batches. Both male and female mice were used. Lung I R-induced endothelial Ca²⁺ signaling and lung dysfunction were not different between male and female mice. TRPV4fl/fl mice, P2Y2Rfl/fl mice, and Panx1fl/fl mice were crossed with Cdh5-CreERT2 mice4 to generate EC-specific knockouts. EC-specific knockout of TRPV4. P2Y2R, and Panx1 was induced by feeding tamoxifen (40 mg/kg/d: Envigo Diet TD.130856) to 6-week-old TRPV4fl/fl Cdh5-Cre, P2Y2Rfl/fl Cdh5-Cre, and Panx1fl/fl Cdh5-Cre mice for 14 days. Mice were used for experiments after a 1-week tamoxifen washout period. Tamoxifen-fed TRPV4fl/fl, P2Y2Rfl/fl, and Panx1fl/fl mice were used as controls. Knockout validation information was provided previously.⁵ C57BL6/J mice were obtained from the Jackson Laboratory (Bar Harbor, ME). Mice were euthanized with pentobarbital (90 mg/kg, i.p.) followed by cranial cervical dislocation.

An independent team member performed random assignment of animals to groups and did not have knowledge of treatment assignment groups. All the experiments were performed in at least two independent batches. The experiments were blinded; information about the groups was withheld from the experimenter.

Genotyping: Genomic DNA was extracted by treating samples of ear tissue with HotSHOT lysis buffer (25 mM NaOH, 0.2 mM EDTA) and then neutralizing with an equal volume of 40 mM Tris-HCL. PCR was performed on a T100 Thermal Cycler (Bio-Rad, Hercules, CA) using 1 unit of Bioline MangoTaq polymerase and buffer (London, England), 1.5 mM MgCl2, 200 μM each dNTP. 1 μM 5′ and 3Y primers, and ~100-250 ng genomic DNA. Reaction products were run on a 1% agarose gel containing 0.2 pg/μL ethidium bromide in TAE buffer (40 mM Tris base. 20 mM acetic acid. 1 mM EDTA) at 90V. Gels were visualized using 302 nm UV light, and the sizes of PCR products were calibrated using a 100-bp DNA Ladder (New England BioLabs, Ipswich, MA). Mice were genotyped using the following primer pairs: Cdh5-Cre, 5′-GCC TGC ATT ACC GGT CGA TGC AAC GA-3′ (forward) (SEQ ID NO: 1) and 5′-GTG GCA GAT GGC GCG GCA ACA CCA TT-3′ (reverse) (SEQ ID NO: 2); TRPV4fl/ft, 5′-CAT GAA ATC TGA CCT CTT GTC CCC-3′ (forward) (SEQ ID NO: 3) and 5′-CGG ACC ACA CGT CTG TCA TGT GTT-3′ (reverse) (SEQ ID NO: 4); and P2Y2Rfl/fl, 5′-TGA CGA CTC AAG ACG GAC AG-3 (SEQ ID NO: 9) (forward) and 5′-TCC CAA CTC ACA CGT ACA AAT G-3′ (reverse) (SEQ ID NO: 5). All primers were obtained from Eurofins Genomics (Louisville, KY). Panx1 genotyping (Panx1fl/fl) was performed by Transnetyx, Inc. (Memphis, TN) using the primer pair, 5′-CAA CAA GTT TGT ACA AA AAA GCA GGC TG-3′ (forward) (SEQ ID NO: 6) and 5′-TGC TTG TAT AGA TAT CTG ACC CAG AGC T-3′ (reverse) (SEQ ID NO: 7), and FAM reporter probe, CCC AGC TTT CTT GTA CAA (SEQ ID NO: 8).

Murine lung IR model: A well-established left lung IRI model involving temporary in vivo ligation of the left hilum (pulmonary artery, vein, and airway) was used.⁶ Briefly, mice were anesthetized with inhaled isoflurane, intubated, ventilated with room air at 150 strokes/min with a tidal volume of 6 mL/g (Harvard Apparatus Co, South Natick, MA), and heparinized (20 U/kg: Hospira Inc, Lake Forest, IL). An anterolateral thoracotomy was performed through the fourth intercostal space to expose the left hilum. A 6-0 Prolene suture (Ethicon. Somerville, NJ) was passed around the left hilum, and the suture ends were passed through a 5-mm length of polyethylene (PE)-50 tubing. A surgical clip was used to fashion a Rummel tourniquet to provide temporary occlusion of the left hilar structures. The thoracotomy was closed, anesthesia was weaned, and analgesia was administered (intraperitoneal buprenorphine. 0.2 mg/kg). Animals recovered in their cages during the 1-hour ischemic period, after which they were anesthetized again and intubated, the Rummel tourniquet was removed, and the thoracotomy was closed. Animals were again extubated, recovered in their cage, and monitored during the 2-hour reperfusion period. Sham animals underwent the same surgical procedure except that application of the Rummel tourniquet to induce ischemia was omitted; thus, these animals were perfused for a total of 3 hours.

Partial pressure of arterial oxygen (PaO₂): After reperfusion, animals were anesthetized, intubated, and ventilated with 100% oxygen. A clamshell thoracotomy was performed, and a 6-0 Prolene suture was passed around the right hilum. The right hilum was ligated, and the tidal volume was decreased to 4 mL/g. After 5 minutes of isolated left lung perfusion/ventilation, a left ventricular puncture was performed with a 25-gauge needle and blood was collected. Left lung-specific PaO₂ was immediately measured using an i-STAT handheld blood analyzer and CG4+ cartridge (Abbott Point of Care, Princeton, NJ).

Lung compliance: Total lung compliance was assessed in separate groups of animals using an IPL-1 isolated perfused lung system (Harvard Apparatus. Holliston, MA) as previously described.⁷ After reperfusion, animals were anesthetized with isoflurane, and a tracheostomy was performed. Animals were ventilated with room air at 150 strokes/min, a tidal volume of 200 mL, and a positive end-expiratory pressure of 2 cm H2O. Animals were exsanguinated by transaction of the inferior vena cava. The pulmonary artery was cannulated through the right ventricle, and a left ventricular incision was made toward the apex of the heart to facilitate venting. Lungs were perfused at 60 mL/g/min with 37° C. Krebs-Henseleit buffer containing 0.20% glucose, 0.014% magnesium sulfate, 0.016% monopotassium phosphate, 0.035% potassium chloride, 0.69% sodium chloride, and 2.1% sodium carbonate. Ventilation and perfusion were maintained for a 5 minute equilibration period. and then lung compliance was recorded using the PULMODYN data-acquisition system (Hugo Sachs Elektronik, March-Hugstetten, Germany).

Bronchoalveolar lavage (BAL) and cytokine analysis: Left lungs were lavaged with 0.4 mL cold phosphate-buffered saline (PBS). The resulting BAL fluid was centrifuged (1200 rpm) at 4° C. for 5 minutes, and the supernatant was stored at −80° C. Cytokine concentrations in BAL fluid were measured using the multiplex Bio-Plex 200 System and Bio-Plex Pro Mouse Cytokine/Chemokine Immunoassays (Bio-Rad, Hercules. CA).

Lung wet-to-dry weight ratio: Lung wet-to-dry weight ratio was measured as an indicator of pulmonary edema. Left lungs of a separate group of animals were explanted after reperfusion and patted dry. Lungs were weighed and then desiccated at 54° C. under vacuum until a stable (dry) weight was obtained. Wet-to-dry weight ratios were calculated.

Neutrophil immunohistochemistry: In separate groups of animals, left lungs were explanted after reperfusion, formalin-fixed via tracheal instillation, paraffin-embedded, and sectioned at 5-μm thickness. Following antigen retrieval in a microwave, neutrophils were identified by immunostaining using a rat anti-mouse neutrophil antibody (LY6B.2; Abcam). Alkaline phosphatase-conjugated anti-rat immunoglobulin G (Sigma-Aldrich) was used as a secondary antibody, and immune signals were detected using Fast-Red (Sigma-Aldrich). Purified normal rat immunoglobulin G (eBioscience, San Diego, CA) was used as a negative control. Sections were counterstained with hematoxylin. For each lung section, neutrophils were counted in five random fields at 20× magnification and averaged. These cell counts did not distinguish between cells in various compartments of the lung (e.g., airspace. interstitial. or marginated) but included all cells in peripheral (alveolar) lung tissue.

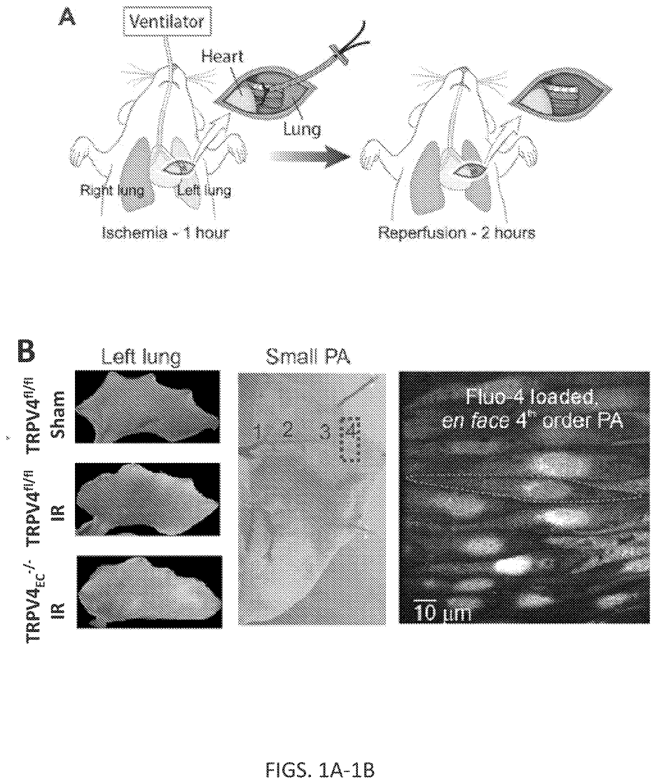

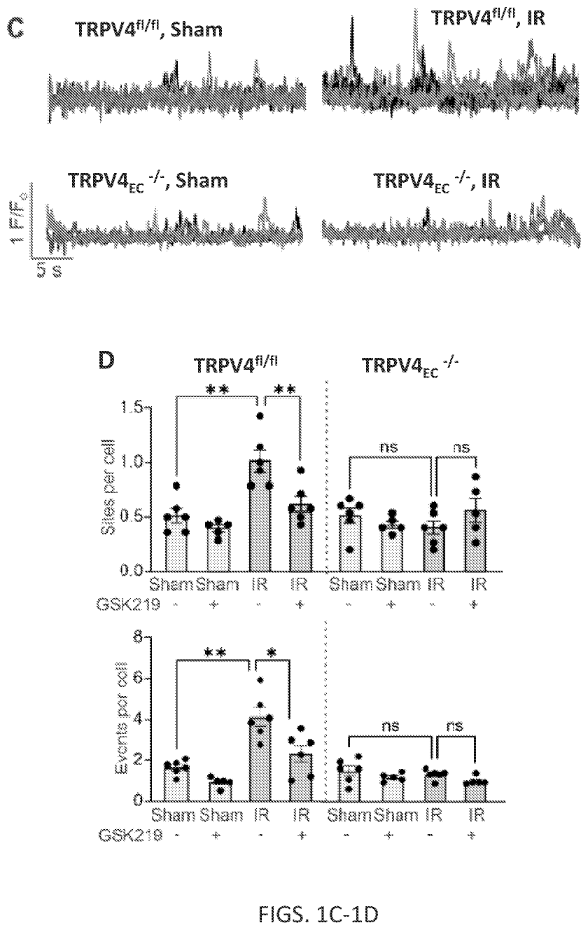

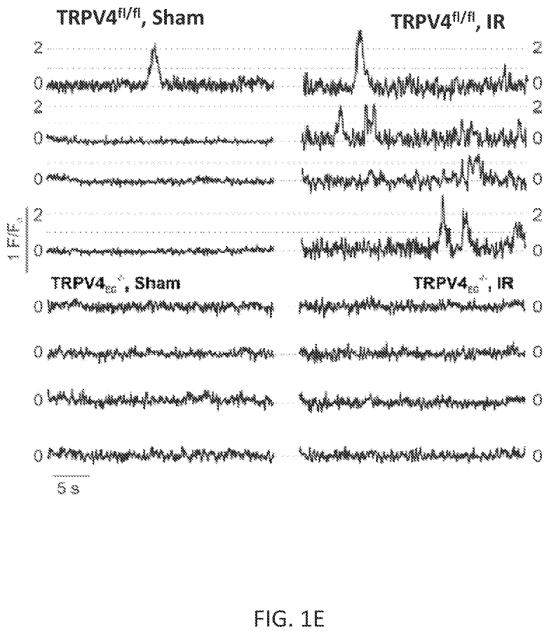

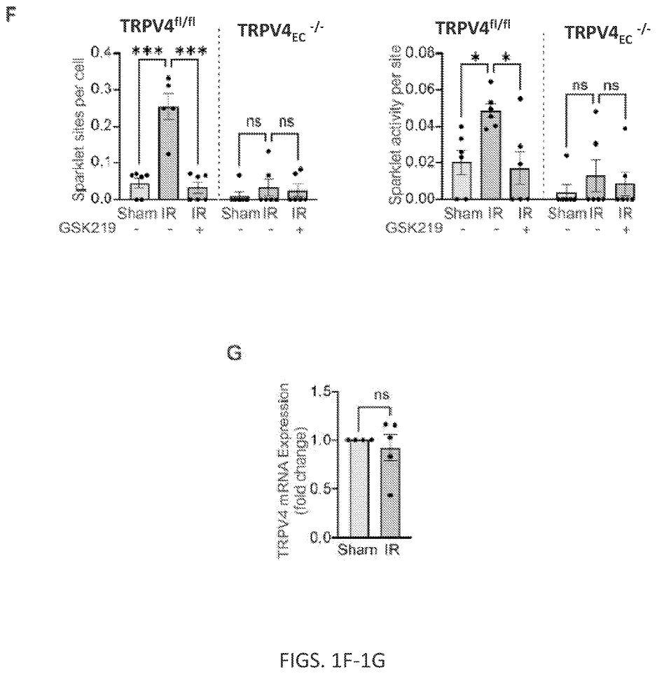

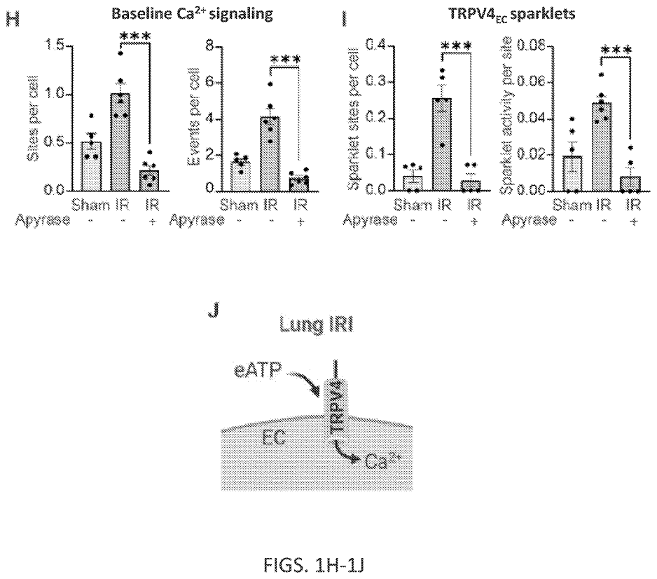

Ca²⁺ imaging in Pas: Ca²⁺ imaging in small Pas was performed as previously described.8 Fourth-order, small (~50 μm diameter) Pas (see FIG. 1B) were dissected from left lungs in cold HEPES-buffered physiological salt solution (HEPES-PSS; 10 mM HEPES, 134 mM NaCl, 6 mM KCl, 1 mM MgCl2 hexahydrate, 2 mM CaCl2) dihydrate, and 7 mM dextrose; pH adjusted to 7.4 using 1 M NaOH). Ca²⁺ imaging studies were performed in en face preparations of small Pas incubated with Fluo-4 AM (10 μM) and pluronic acid (0.04%) at 37° C. for 10 minutes. For studies after sham or lung IR surgery, small Pas were immediately dissected out after the indicated reperfusion time and incubated en face with Fluo-4 AM. For acute hypoxia-reoxygenation (HR) exposure, small Pas were washed with PBS, after which serum free/glucose-free endothelial cell medium was added. Small Pas were placed in a humidified, sealed hypoxia chamber (Billups-Rothenberg, San Diego, CA), which was subsequently flushed with 95% nitrogen/5% CO2 for 20 minutes and placed in a 37° C. incubator to initiate hypoxia. After 1 hour of hypoxia, the chamber was opened to terminate hypoxia. Pas were then washed with PBS; endothelial growth medium containing 10 μM Fluo-4 AM at 37° C. was added. and Pas were reoxygenated by placing in a 37° C., humidified 5% CO2 incubator for 10 minutes. Ca²⁺ images were acquired at 30 frames per second using an Andor Revolution WD (with Borealis) spinning-disk confocal imaging system (Andor Technology, Belfast, UK) comprising an upright Nikon microscope with a 60× water-dipping objective (numerical aperture [NA], 1.0) and an electron-multiplying CCD camera (iXon888), as was done previously.⁹ Pas were superfused with PSS (pH 7.4), and all experiments were performed at 37° C. Fluo-4 was excited using a 488-nm solid-state laser, and emitted fluorescence was captured using a 525/36-nm band-pass filter. Pas were treated with the sarco-endoplasmic reticulum (SR/ER) Ca²⁺-ATPase inhibitor cyclopiazonic acid (CPA; 20 μM) for 10 minutes at 37° C. prior to imaging to eliminate intracellular Ca²⁺-release signals. respectively. We previously showed that CPA per se does not alter the activity of TRPV4 sparklets in endothelial cells. The effects of GSK219, ARC, and apyrase on pulmonary endothelial Ca²⁺ signals were studied 10 minutes after the addition of these drugs. Baseline Ca²⁺ signaling activity was recorded in the absence of CPA.

Analysis of baseline Ca²⁺ signaling activity and TRPV4 sparklet activity in the pulmonary endothelium: Ca²⁺ images acquired using the procedure described above were analyzed with custom-designed SparkAn software (source code available at https://github.com/vesselman/SparkAn).¹⁰ Fractional fluorescence (F/F0) traces were obtained by placing a 1.7 μm2 (5×5 pixels) region of interest (ROI) at the peak event amplitude. Representative F/F0 traces were filtered using a Gaussian filter and a cutoff corner frequency of 4 Hz. Overall baseline Ca²⁺ signals were automatically detected using a threshold of 1.25 F/F0. Changes in baseline (e.g., due to movement of the artery or a change in focus) were accounted for by obtaining a continuous baseline using a running-average approach from start to end, 19 points at a time, and then dividing the F/F0 trace by the continuous baseline. Each trace shown in figures indicates Ca²⁺ signaling activity at a single site.

TRPV4 sparklets were assessed as increases in fluorescence relative to baseline fluorescence, obtained by averaging 10 quiescent images prior to stimulation. TRPV4 Ca²⁺ sparklets were assessed based on previously established methods.^{9,11} Average TRPV4 sparklet activity is defined as NP_{O}, where N is the number of TRPV4 channels per site and P_{O} is the open-state probability of the channel. NP_{O} was calculated using the Single Channel Search module of Clampfit. quantal amplitudes derived from all-points histograms (0.3 AF/F0 for Fluo-4-loaded Pas),¹² and the following equation: NPO=(Tlevel1+2Tlevel2+3Tlevel3+4Tlevel4)/Ttotal, where T represents the dwell time at each quantal level and Ttotal is the total recording duration. Average NP_{O} per site was obtained by averaging the NP_{O} for all sites in a field of view (110 μm×110 μm). NP_{O} values per site from all fields in an artery were averaged to obtain NP_{O} per site for that artery. The total number of sites per cell corresponds to all sparklet sites in a field divided by the number of cells in that field. Sparklet sites per cell were averaged for all fields in an artery to obtain sparklet sites per cell for that artery.

Exposure of PMVECs to acute HR and Ca²⁺ imaging in PMVECs: Primary human and murine pulmonary microvascular endothelial cells (PMVECs; Cell Biologics, Chicago, IL) were cultured on gelatin-coated flasks in endothelial cell medium (Cell Biologics). For acute HR exposure, PMVECs were washed with PBS, and serum-free/glucose-free endothelial cell medium was added. PMVECs were placed in a humidified, sealed hypoxia chamber (Billups-Rothenberg, San Diego, CA), whereupon the chamber was flushed with 95% nitrogen/5% CO2 for 20 minutes and placed in a 37° C. incubator to initiate hypoxia. After 1 hour of hypoxia, the chamber was opened to terminate hypoxia. Cells were washed with PBS; complete endothelial growth medium containing 0.1 μM Calbryte 520 AM (AAT Bioquest, Pleasanton, CA) was added; and cultures were incubated at 37° C. in a humidified 5% CO2 incubator (reoxygenation) for 10 minutes. Control, normoxic PMVECs underwent the same procedure except that the chamber was not flushed with nitrogen/CO2.

Ca²⁺ imaging in human and mouse PMVECs was performed using an Andor Dragonfly 505 spinning-disk confocal imaging system with Borealis illumination and an Integrated Laser Engine solid-state laser source (Andor Technology, Belfast, UK), coupled with a Leica Dmi8 inverted microscope (Leica) and 63× objective (NA 1.47)¹³ Images were acquired at 30 frames per second using Fusion 2.3 (Oxford Instruments, Belfast, UK) and an electron-multiplying CCD camera (iXon888). Calbryte 520 AM was excited at 488 nm, and emitted fluorescence was captured using a 525/50 bandpass emission filter. Image analysis was performed as described above for small Pas.

Whole-cell patch-clamp electrophysiology: Patch-clamp electrodes were pulled with a Narishige PC-100 puller (Narishige International USA, Inc, Amityville, NY) and polished using a MicroForge MF-830 polisher (Narishige International. USA). The pipette resistance was 4-6 MΩ. Data were acquired using a Multiclamp 700B amplifier connected to a Digidata 1550B system and analyzed using Clampfit 11.1 software (Molecular Devices, San Jose, CA). TRPV4 channel currents were recorded from human and murine PMVECs. GSK101 (10 nM)-induced outward currents through TRPV4 channels were assessed using the whole-cell patch-clamp configuration in the presence of ruthenium red (RuR), included to prevent Ca⁺ influx through TRPV4 channels and activation of potassium channels.^{11,14} The intracellular solution consisted of 10 mM HEPES, 30 mM KCl, 10 mM NaCl, 110 mM K-aspartate, and 1 mM MgCl2 (adjusted to pH 7.2 with NaOH). HEPES-PSS was used as the bath solution. TRPV4 currents were measured using a voltage-clamp protocol in which voltage-ramp pulses (−100 mV to +100 mV) were applied over 200 ms from a holding potential of −50 mV. Currents were measured before and 5 minutes after treatment with GSK101 (10 nmol/L) followed by GSK219 (100 nmol/L), with GSK219-sensitive currents defined as TRPV4 channel-mediated currents.

Quantitative polymerase chain reaction (qPCR): Endothelial cell (EC) sheets were used for mRNA isolation, as described previously.⁹ Briefly, small Pas were enzymatically digested by incubating in dissociation solution (55 mM NaCl, 80 mM Na-glutamate, 6 mM KCl, 2 mM MgCl2, 0.1 mM CaCl2), 10 mM glucose, 10 mM HEPES, pH 7.3) containing 1 mg/mL neutral protease (Worthington, Freehold, NJ) for 30 minutes at 37° C. Following enzymatic digestion. EC sheets were gently squeezed out from small Pas with fine tips of 1-mL syringe needles. EC sheets were placed in a perfusion chamber and allowed to settle for 15 minutes at room temperature. EC sheets were collected with a micropipette (~0.5-0.8 MΩ) by applying gentle suction. The purity of Ecs (>95%) was confirmed in control experiments by immunostaining with FITC-tagged CD31.8 Isolated Ecs (~2000) were transferred to TRI Reagent (Zymo Research, CA) and homogenized using a Standard Micro-homogenizer (PRO Scientific Inc., Oxford, CT). Total RNA was isolated using a Direct-zol RNA Miniprep Kit (Zymo Research, CA), treated with an on-column DNA removal protocol, and converted to cDNA using an iScript cDNA Synthesis Kit (Bio-Rad, Hercules. CA). qPCR was performed on a Bio-Rad CFX96 qPCR Detection System using a reaction mix containing Bio-Rad 2×SYBR Green Master Mix, 200 nM 5′ and 3′ primers, and 20 nM cDNA. qPCR primers for Panx1, TRPV4, and GAPDH (internal control) were obtained from GeneCoepia Inc. (Rockville, MD). P2Y2R qPCR primers were obtained from Bio-Rad (Hercules, CA). Results were analyzed using the ΛΛCt method.

Luciferase assay for total ATP release: Small Pas were cut open and pinned down en face on a Sylgard block. Pas were then placed in black, opaque 96-well plates and incubated in HEPES-PSS for 10 minutes at 37° C., followed by incubation with the ectonucleotidase inhibitor ARL 67156 (300 μmol/L; Tocris Bioscience, Minneapolis, MN) for 30 minutes at 37° C. A 50-μL aliquot of each sample was transferred to another black, opaque 96-well plate, and ATP was measured using an ATP Bioluminescence HSII kit (Roche Applied Science, Penzberg, Germany).⁵ Briefly, 50 μL of the luciferase reagent, luciferin, provided in the assay kit (Roche Applied Science), was added to each well and. following a 5-second orbital mix, luminescence was recorded using a luminometer (FluoStar Omega). ATP concentrations were calculated by reference to an ATP standard curve.

Studies in freshly isolated small Pas from human lungs. Deidentified human lung tissue sample was obtained from normal-looking areas of explanted lung during a lung transplantation, in accordance with policies of the University of Virginia Institutional Review Board (IRB #19044). Multiple small Pas (~50 m) were dissected and used for Ca²⁺ imaging experiments, as described previously.⁸

Statistical analysis: Results are presented as means±SEM. An n-value of 1 was defined as one small PA in imaging experiments (Ca²⁺ imaging), one cell in patch-clamp experiments, and one mouse for ATP measurements, qPCR experiments, lung function, lung edema, neutrophil infiltration, and inflammatory cytokine assays. Data were obtained from at least three mice in experiments performed in at least two independent batches. Individual data points are shown for each dataset. Graphical representations of all data were prepared using CorelDraw Graphics Suite X7 (Ottawa, ON, Canada), and all data were statistically analyzed using GraphPad Prism 8.3.0 (Sand Diego, CA). Power analyses used to determine group sizes and study power (>0.8) were performed using GLIMMPSE software (α=0.05; >20% change). Using this method, we estimated at least five cells per group for patch-clamp experiments, five small Pas per group for imaging experiments, and five mice for qPCR, ATP, lung function, neutrophil, and cytokine measurements. Data were analyzed using two-tailed, paired or independent t-test for comparisons of data collected from two different treatments, and one- or two-way ANOVA to investigate statistical differences among more than two different treatments. A Tukey correction was applied for multiple comparisons with one-way ANOVA, and a Bonferroni correction was performed for multiple comparisons with two-way ANOVA. Statistical significance was defined as a p-value<0.05.

### METHODS BIBLIOGRAPHY

1. Moore C, Cevikbas F, Pasolli H A, Chen Y, Kong W, Kempkes C, Parekh P, Lee S H, Kontchou N A, Yeh I, et al. UVB radiation generates sunbum pain and affects skin by activating epidermal TRPV4 ion channels and triggering endothelin-1 signaling. Proc Natl Acad Sci USA. 2013; 110:E3225-3234. Doi: 10.1073/pnas.1312933110
2. Chen X. Qian S, Hoggatt A, Tang H. Hacker T A, Obukhov A G, Herring P B, Seye Cl. Endothelial Cell-Specific Deletion of P2Y2 Receptor Promotes Plaque Stability in Atherosclerosis-Susceptible ApoE-Null Mice. Arterioscler Thromb Vasc Biol. 2017; 37:75-83. Doi: 10.1161/ATVBAHA.116.308561
3. Billaud M, Chiu Y H, Lohman A W, Parpaite T, Butcher J T, Mutchler S M, DeLalio L J, Artamonov M V, Sandilos J K, Best A K, et al. A molecular signature in the pannexinl intracellular loop confers channel activation by the alphal adrenoreceptor in smooth muscle cells. Science signaling. 2015; 8:ra17. Doi: 10.1126/scisignal.2005824
4. Sorensen I, Adams R H, Gossler A. DLL1-mediated Notch activation regulates endothelial identity in mouse fetal arteries. Blood. 2009; 113:5680-5688. Doi: 10.1182/blood-2008-08-174508
5. Daneva Z, Ottolini M. Chen Y L, Klimentova E, Kuppusamy M, Shah S A, Minshall R D, Seye C L, Laubach V E, Isakson B E. et al. Endothelial pannexin 1-TRPV4 channel signaling lowers pulmonary arterial pressure in mice. Elife. 2021; 10. Doi: 10.7554/eLife.67777
6. Yang Z, Sharma A K, Marshall M, Kron I L, Laubach V E. NADPH oxidase in bone marrow-derived cells mediates pulmonary ischemia-reperfusion injury. Am J Respir Cell Mol Biol. 2009:40:375-381. Doi: 10.1165/rcmb.2008-03000C
7. Haywood N, Ta H Q, Zhang A, Charles E J, Rotar E, Noona St, Salmon M, Daneva Z, Sonkusare S K, Laubach V E. Endothelial Transient Receptor Potential Vanilloid 4 Channels Mediate Lung Ischemia-Reperfusion Injury. Ann Thorac Surg. 2022; 113:1256-1264. Doi: 10.1016/j.athoracsur.2021.04.052
8. Daneva Z. Marziano C. Ottolini M. Chen Y L, Baker™, Kuppusamy M, Zhang A, Ta H Q, Reagan C E, Mihalek A D, et al. Caveolar peroxynitrite formation impairs endothelial TRPV4 channels and elevates pulmonary arterial pressure in pulmonary hypertension. Proc Natl Acad Sci USA. 2021; 118. Doi: 10.1073/pnas.2023130118
9. Ottolini M. Hong K. Cope E L, Daneva Z, DeLalio U. Sokolowski J D. Marziano C. Nguyen N Y, Altschmied J. Haendeler J, et al. Local peroxynitrite impairs endothelial transient receptor potential vanilloid 4 channels and elevates blood pressure in obesity. Circulation. 2020; 141:1318-1333. Doi: 10.1161/CIRCULATIONAHA.119.043385
10. Chen Y L, Daneva Z. Kuppusamy M, Ottolini M, Baker™, Klimentova E, Shah S A, Sokolowski J D, Park M S, Sonkusare S K. Novel Smooth Muscle Ca(2+)-Signaling Nanodomains in Blood Pressure Regulation. Circulation. 2022; 146:548-564. Doi: 10.1161/CIRCULATIONAHA.121.058607
11. Sonkusare S K, Dalsgaard T, Bonev A D, Hill-Eubanks D C, Kotlikoff M I, Scott J D, Santana L F, Nelson M T. AKAP150-dependent cooperative TRPV4 channel gating is central to endothelium-dependent vasodilation and is disrupted in hypertension. Science signaling. 2014; 7:ra66. Doi: 10.1126/scisignal.2005052
12. Marziano C, Hong K. Cope E L, Kotlikoff M I, Isakson B E, Sonkusare S K. Nitric Oxide-Dependent Feedback Loop Regulates Transient Receptor Potential Vanilloid 4 (TRPV4) Channel Cooperativity and Endothelial Function in Small Pulmonary Arteries. J Am Heart Assoc. 2017; 6. Doi: 10.1161/JAHA.117.007157
13. Daneva Z, Chen Y L, Ta H Q, Manchikalapudi V, Bazaz A, Laubach V E, Sonkusare S K. Endothelial I K and SK channel activation decreases pulmonary arterial pressure and vascular remodeling in pulmonary hypertension. Pulm Circ. 2023; 13:e12186. Doi: 10.1002/pul2.12186
14. Sonkusare S K, Bonev A D, Ledoux J, Liedtke W, Kotlikoff M I, Heppner T J, Hill-Eubanks D C, Nelson M T. Elementary Ca²⁺ signals through endothelial TRPV4 channels regulate vascular function. Science. 2012; 336:597-601. Doi: 10.1126/science.1216283

### Results

Lung I R increases the activity of TRPV4EC channels via eATP.

Based on our findings that TRPV4EC−/− mice are protected against lung IRI,¹⁶ we hypothesized that increased Ca²⁺ influx through TRPV4EC channels mediates lung IRI. To test this, we used a well-established left-lung hilar-ligation model of IRI in mice (FIG. 1A) involving 1 hour of ligation (ischemia) followed by 2 hours of reperfusion.^{9,20} After I R, mice display significant lung dysfunction, neutrophil infiltration, and increased levels of inflammatory cytokines in bronchoalveolar lavage (BAL) fluid.¹⁶ Control mice underwent sham surgery with no hilar ligation. The activity of individual Ca²⁺ signals in intact pulmonary endothelium was recorded in an en face preparation of small pulmonary arteries (Pas; ~50 μm diameter with single smooth muscle cell layer;²¹ FIG. 1B) loaded with Fluo-4 AM (10 μM) using high-speed spinning-disk confocal Ca²⁺ imaging (FIG. 1B). Baseline Ca²⁺ signaling activity (number of Ca²⁺ event sites per EC and number of Ca²⁺ events per EC) was higher in the endothelium from mice exposed to IR compared with that in sham mice (FIG. 1C, left). Our previous studies showed that baseline activity of Ca²⁺ signals in the PA endothelium primarily consists of inositol triphosphate receptor (IP3R)-mediated Ca²⁺ release from intracellular stores and Ca²⁺ influx through TRPV4 channels.^{10,11,22} The specific TRPV4 channel inhibitor, GSK2193874 (100 nM: hereafter, GSK219), completely abolished the increase in baseline endothelial Ca²⁺ signals after lung IR in control mice (FIG. 1D). Moreover, the lung IR-induced increase in endothelial Ca²⁺ signaling activity was lost in small Pas from TRPV4EC−/− mice (FIGS. 1C and 1D). These results suggest that increased TRPV4EC channel activity mediates IR-induced increases in endothelial Ca²⁺ signals.

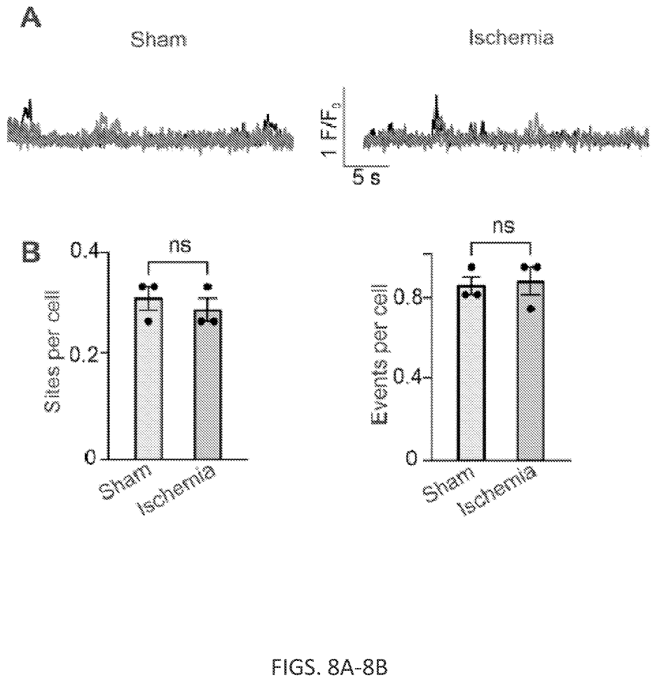

To verify this possibility, we studied TRPV4EC channel activity in the form of TRPV4EC sparklets²³ (individual Ca²⁺-influx signals through TRPV4EC channels) in the intact endothelium from small Pas of mice that underwent lung IR or sham surgery. TRPV4EC sparklets were recorded in the presence of 20 μM cyclopiazonic acid (CPA: sarco/endoplasmic reticulum ATPase [SERCA] inhibitor) to eliminate interference from intracellular Ca²⁺-release signals. CPA, by itself, does not alter the activity of TRPV4 channels.^{19,24} TRPV4EC sparklet activity, both sparklet sites per cell and activity per site, was increased in mice exposed to IR compared with those receiving sham surgery (FIGS. 1E and 1F). The IR-induced increase in TRPV4EC sparklet activity was inhibited by the TRPV4 inhibitor GSK219 and was absent in the endothelium from TRPV4EC−/− mice (FIGS. 1E and 1F). In small Pas from mice that underwent 1-hour of left lung ischemia but not reperfusion, endothelial Ca²⁺ signaling activity was not altered (FIGS. 8A-8B), suggesting that reperfusion was required for IR-induced increase in pulmonary endothelial Ca²⁺ signaling. These results provide the first direct evidence that lung IR increases the activity of TRPV4EC channels in the pulmonary endothelium.

Next, we tested the possibility that increased expression of TRPV4EC channels underlies the increase in channel activity after IR. A quantitative polymerase chain reaction (qPCR) analysis of freshly isolated Ecs from small Pas revealed that TRPV4 mRNA levels were not different between lung IR and sham groups (FIG. 1G), implying that increased TRPV4EC channel activity after lung IR is not attributable to increased channel expression. In this regard. recent studies suggest that eATP is an endogenous activator of TRPV4EC channels in the intact pulmonary endothelium.⁸ Hydrolyzing eATP with apyrase (10 U/mL) abolished IR-induced increases in baseline Ca²⁺ signaling activity and TRPV4EC channel activity in small Pas (FIGS. 1H and 1I), identifying eATP as a novel mediator of increased TRPV4EC channel activity after lung IR (FIG. 1J).

Endothelial P2Y2R signaling activates TRPV4 channel-dependent Ca²⁺ influx in lung IR.

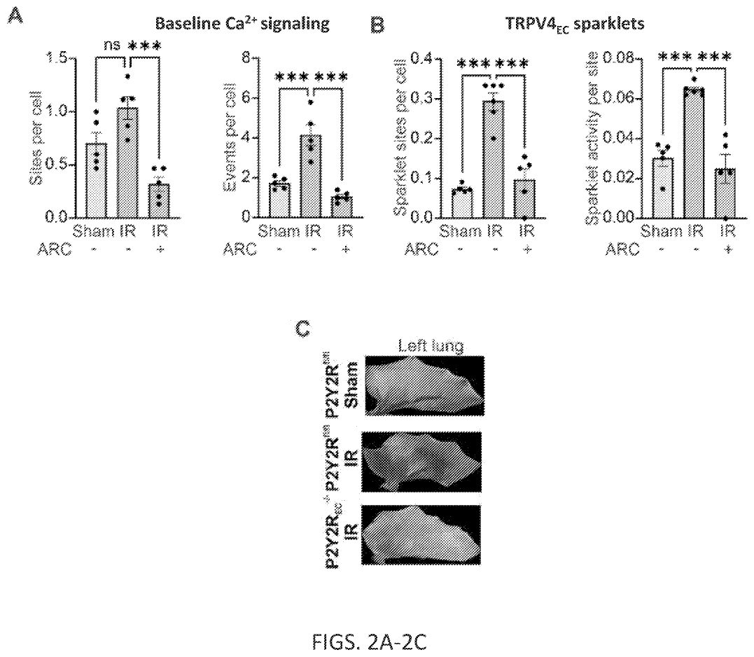

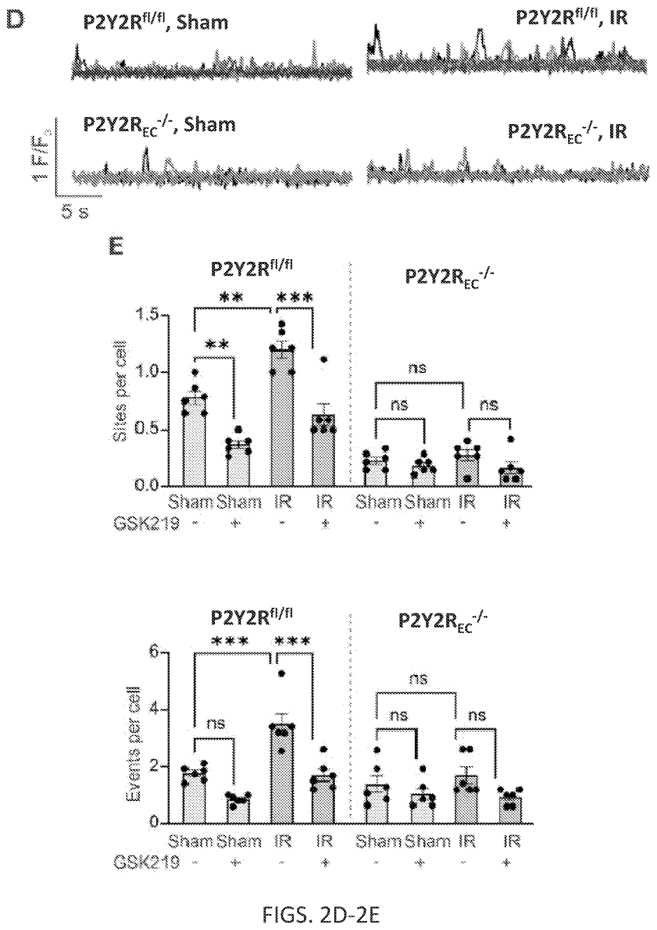

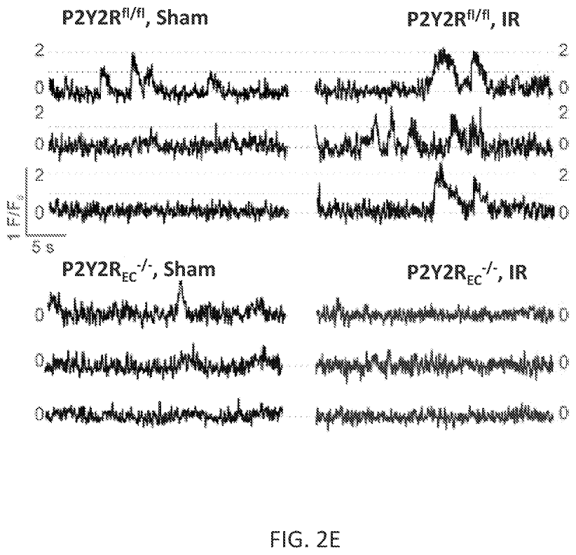

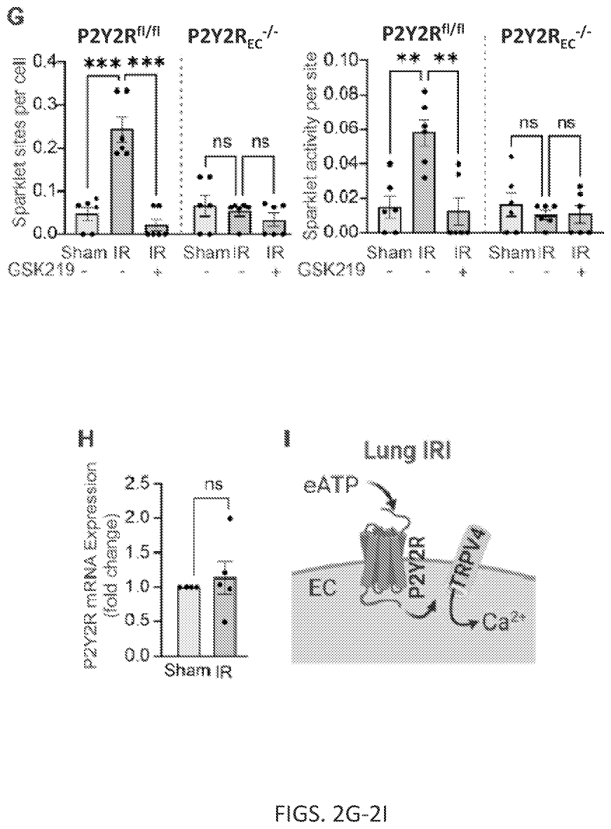

We earlier showed that, under healthy conditions, eATP activates TRPV4EC channels through purinergic P2Y2R signaling in small Pas.⁸ Therefore, we used the P2Y2R inhibitor, AR-C 118925XX (ARC; 10 μmol/L), to test the role of P2Y2Rs in lung IR-induced increases in TRPV4EC channel activity in small Pas. Treatment with ARC abolished lung IR-induced increases in baseline endothelial Ca²⁺ signaling activity in small Pas (FIG. 2A). ARC also inhibited lung IR-induced increases in TRPV4EC sparklet activity (FIG. 2B). To test whether endothelial P2Y2Rs mediate TRPV4EC channel activation after lung IR, we used tamoxifen-inducible, endothelium-specific P2Y2R-knockout (P2Y2REC−/−) mice.⁸ P2Y2REC−/− and control (P2Y2Rfl/fl) mice underwent lung IR or sham surgeries. IR-induced gross injury was apparent in left lungs from P2Y2Rfl/fl mice, but not in P2Y2REC−/− mice (FIG. 2C). Baseline Ca²⁺ signaling activity (FIGS. 2D and 2E) and TRPV4EC sparklet activity (FIGS. 2F and 2G) were increased in control mice after IR compared with those after sham surgery. IR-induced increases in baseline Ca²⁺ signaling activity and TRPV4EC sparklet activity were absent in the endothelium from P2Y2REC−/− mice (FIGS. 2E-2G). Moreover, lung IR did not alter the expression of endothelial P2Y2Rs at the mRNA level (FIG. 2H), implying that lung IRI involves increased activation, not upregulation, of endothelial P2Y2Rs. These data provide strong evidence that eATP acts through P2Y2REC to activate TRPV4EC channels in lung IRI (FIG. 2I).

Endothelial P2Y2R deletion attenuates lung IRI.

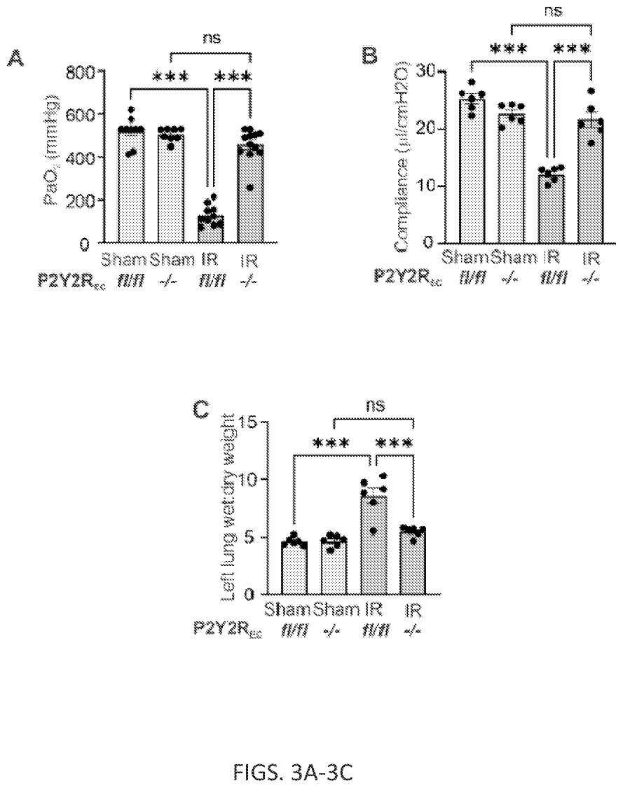

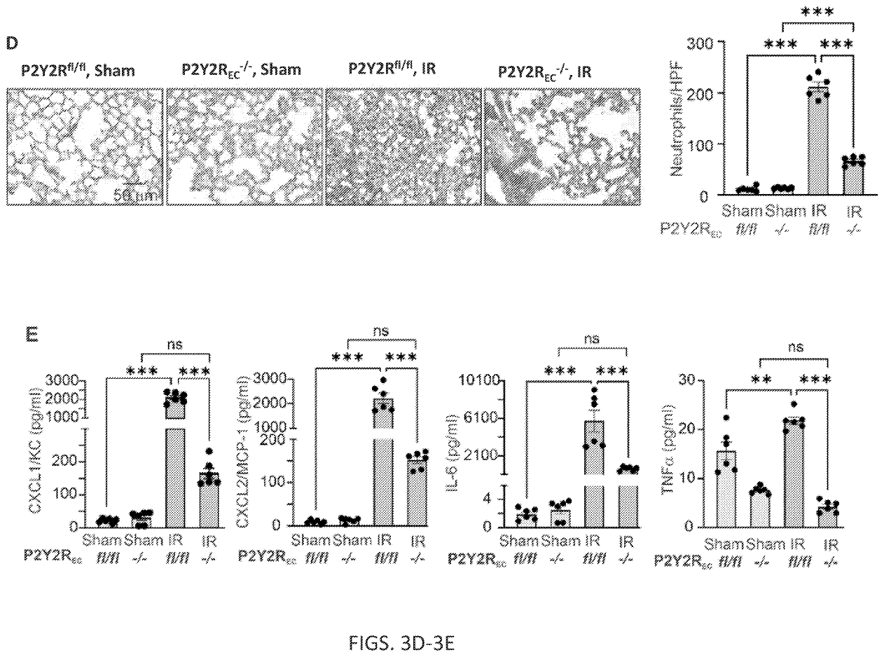

Compared with sham mice, control P2Y2Rfl/fl mice exposed to IR developed significant pulmonary dysfunction, as measured by reduced partial pressure of oxygen (PaO₂) and lung compliance (FIGS. 3A and 3B). P2Y2REC−/− mice undergoing IR exhibited significantly attenuated lung dysfunction after IR, as demonstrated by improved PaO₂ and compliance (FIGS. 3A and 3B). Significant pulmonary edema, as measured by lung wet-to-dry weight ratio, occurred in P2Y2Rfl/fl mice after IR, which was prevented in P2Y2REC−/− mice (FIG. 3C), suggesting preservation of the vascular endothelial barrier. The lung endothelial barrier was also assessed by measuring neutrophil infiltration (which requires migration across the endothelial barrier) and pro-inflammatory cytokine levels in BAL fluid. Neutrophil infiltration in lung tissue was markedly increased in control P2Y2Rfl/fl mice after IR (FIG. 3D), consistent with impaired endothelial barrier function. IR-induced neutrophil infiltration was significantly reduced in lungs from P2Y2REC−/− mice (FIG. 3D), providing evidence that endothelial P2Y2R deletion preserves endothelial barrier function after lung IR. Moreover, P2Y2Rfl/fl mice undergoing IR exhibited significantly higher levels of proinflammatory cytokines, including chemokine (C—X—C motif) ligand 1 (CXCL1/KC), chemokine (C—X—C motif) ligand 2 (CXCL2/MCP-1), interleukin 6 (IL-6), and tumor necrosis factor alpha (TNFα) (FIG. 3E). Expression of these cytokines was significantly reduced in P2Y2REC−/− mice after lung IR. Collectively, these results supported a role for endothelial P2Y2Rs in lung IR-induced pulmonary dysfunction, edema, inflammation, and endothelial barrier disruption.

P2Y2R inhibition reduces proinflammatory cytokine release from pulmonary endothelial cells in a surrogate model of lung IR.

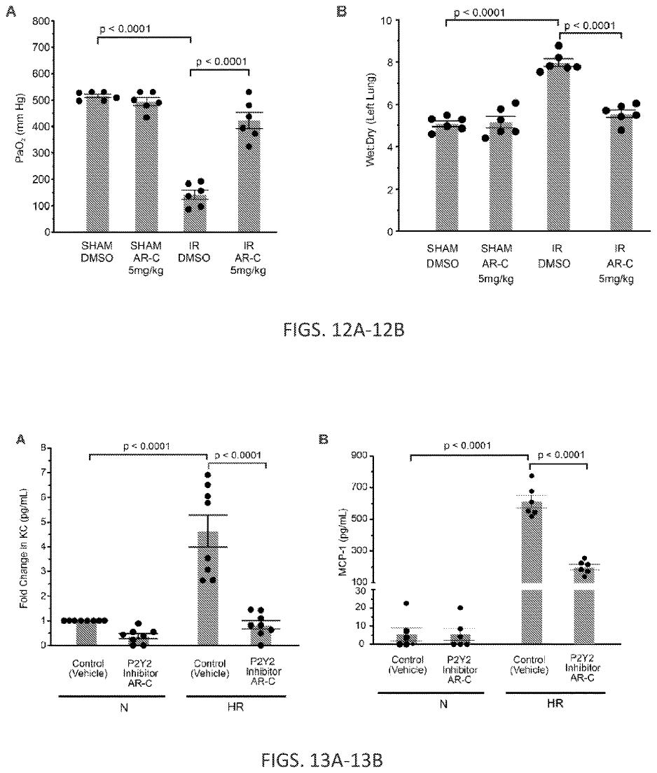

We used 6 hours hypoxia followed by 4 hours normoxia/reoxygenation (HR) in murine primary pulmonary microvascular endothelial cells (PMVECs) as a surrogate model of lung IR. Control cells were exposed to 10 hours normoxia (N). PMVECs were treated with vehicle or P2Y2R inhibitor AR-C (10 mM, 6 hrs). The concentrations of chemokine (C—X—C motif) ligand 1 (CXCL1) and monocvte chemoattractant protein-1 (MCP-1) in the culture media was measured using a mouse keratinocyte-derived chemokine (KC)/CXCL1 Quantikine ELISA kit (R&D Systems, Minneapolis, MN) and multiplex cytokine panel assay (Bio-Rad Laboratories, Hercules, CA), respectively. KC and MCP-1 levels were increased in PMVECs exposed to HR. Treatment of PMVECs with P2Y2R inhibitor AR-C reduced the levels of KC (FIG. 13A) and MCP-1 (FIG. 13B) in the culture media. These data show that P2Y2R inhibition has beneficial effects by lowering the release of proinflammatory cytokines from PMVECs following HR.

Treatment with P2Y2R inhibitor has beneficial effects on lung function and lung edema after lung IR.

In a mouse model of lung IRI (left-lung hilar-ligation, involving 1 hour of ligation (ischemia) followed by 2 hours of reperfusion), the mice were injected with vehicle (DMSO) or P2Y2R inhibitor AR-Cl 18925XX (AR-C, 5 mg/kg, 1 hr prior to left-lung hilar ligation, injected via jugular vein). Partial pressure of oxygen (PaO₂), measured in an ex vivo lung perfusion setup, was used as an indicator of lung function. Compared with sham mice, mice exposed to IR showed a significant lung dysfunction, as measured by reduced PaO₂ (FIG. 12A). Notably, pre-treatment of mice exposed to IR with P2Y2R inhibitor AR-C reduced lung dysfunction, as demonstrated by improved PaO₂ (FIG. 12A). Pulmonary edema, as measured by the lung wet-to-dry weight ratio, occurred in mice after IR exposure. but not in mice pre-treated with P2Y2R inhibitor AR-C before exposure to IR (FIG. 12B), suggesting preservation of the vascular endothelial barrier after P2Y2R inhibition. These data show that P2Y2R inhibition has beneficial effects on lung function and vascular endothelial barrier after lung IR.

Acute hypoxia-reoxygenation (HR) induces P2Y2R-TRPV4 signaling in the pulmonary endothelium.

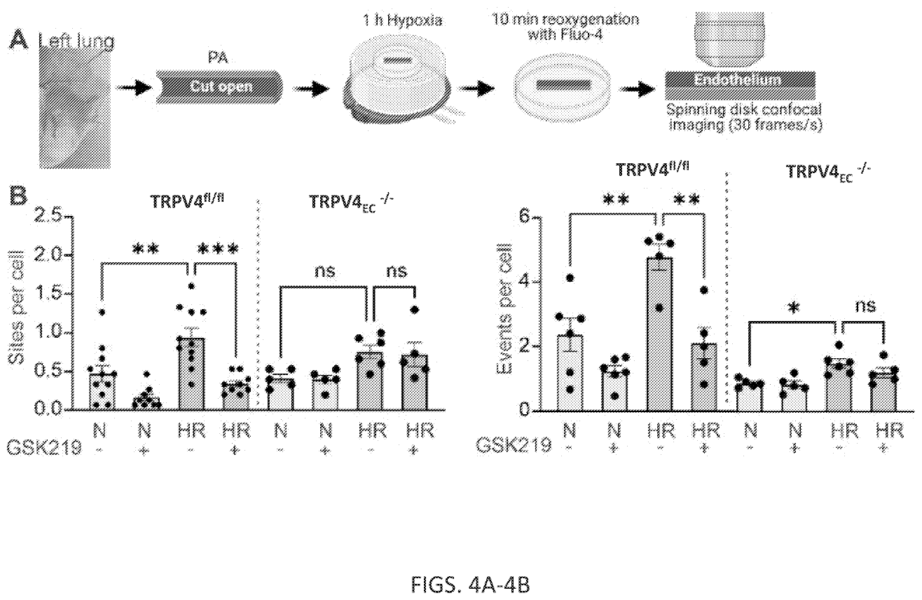

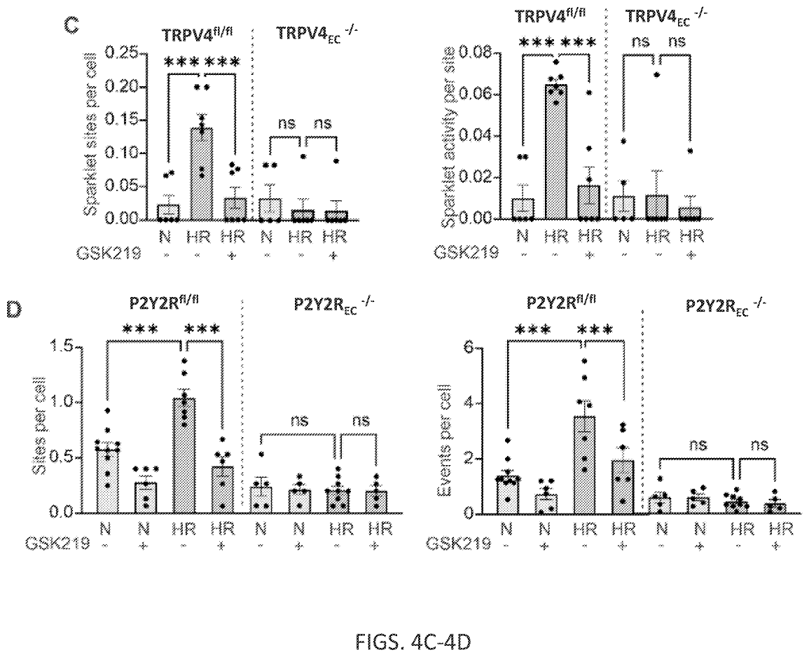

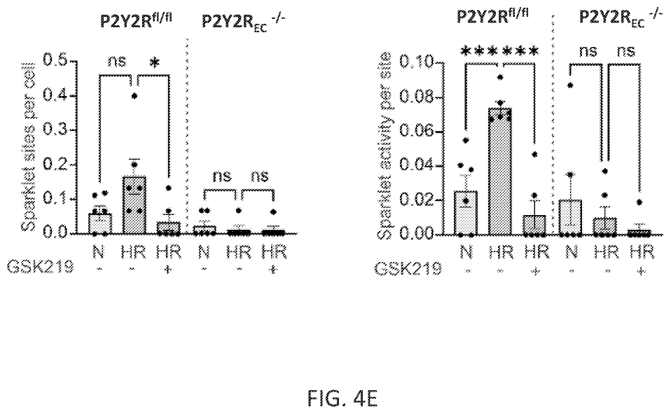

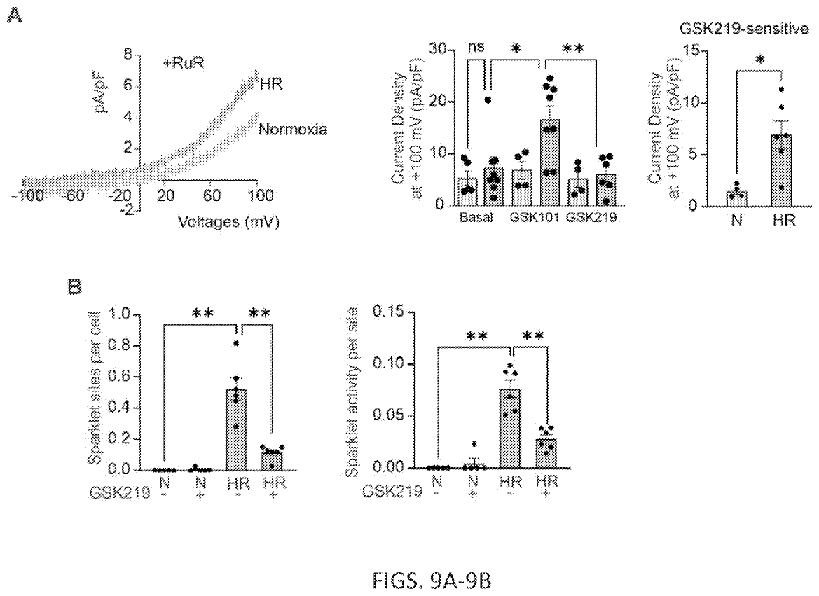

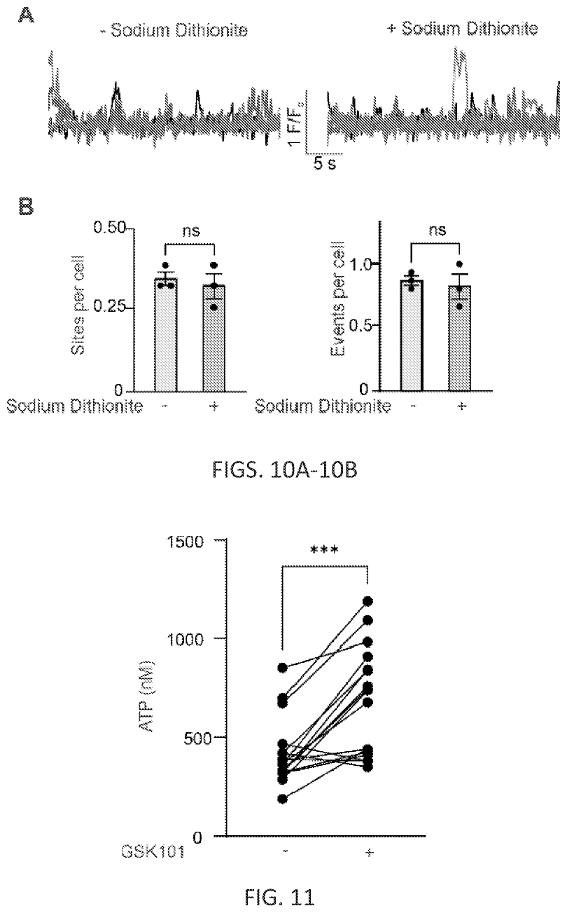

To determine whether IR-induced increases in P2Y2REC-TRPV4EC signaling are a result of hypoxia (ischemia) followed by reoxygenation (reperfusion), we used a novel ex vivo surrogate model (FIG. 4A) of IR, in which small Pas from TRPV4EC−/−. P2Y2REC−/−, and respective control mice are exposed to 1-hour hypoxia followed by 10 minutes of reoxygenation (HR) or to normoxia for 70 minutes. The Ca²⁺ indicator, Fluo-4 AM (10 μM), was included during the final 10 minutes of incubation for each group. Acute H R exposure increased the activity of baseline endothelial Ca²⁺ signaling (FIG. 4B) and TRPV4 sparklets (FIG. 4C) in small Pas from TRPV4fl/fl mice compared with that observed following exposure to normoxia. Small Pas from TRPV4EC−/− mice were protected against HR-induced increases in baseline endothelial Ca²⁺ signaling and TRPV4EC sparklet activity. Moreover, treatment with the TRPV4 inhibitor, GSK219 (100 nM), completely abolished the increase in endothelial Ca²⁺ signals in small Pas from TRPV4fl/fl mice exposed to HR (FIGS. 4B and 4C). Similar to the case for the lung IR model, HR-induced increases in endothelial Ca²⁺ signaling (FIG. 4D) and TRPV4 sparklet (FIG. 4E) activity were absent in small Pas from P2Y2REC−/− mice. HR also increased baseline Ca²⁺ signaling and TRPV4 sparklets in vitro in murine primary pulmonary microvascular endothelial cells (PMVECs), in association with an increase in ionic currents through TRPV4 channels (FIGS. 9A-9B), indicating that direct exposure to acute HR increases TRPV4EC activity in small Pas as well as PMVECs. Hypoxia alone (induced using sodium dithionite, 1 mM)²⁵ did not alter baseline endothelial Ca²⁺ signaling activity in small Pas (FIGS. 10A-10B), suggesting that reoxygenation was required for HR-induced increase in pulmonary endothelial Ca²⁺ signals. Collectively, these results suggest that acute HR exposure increases TRPV4EC channel activity in the pulmonary endothelium through P2Y2R signaling.

Acute H R exposure increases TRPV4 channel activity in a P2Y2R-dependent manner in human pulmonary Ecs.

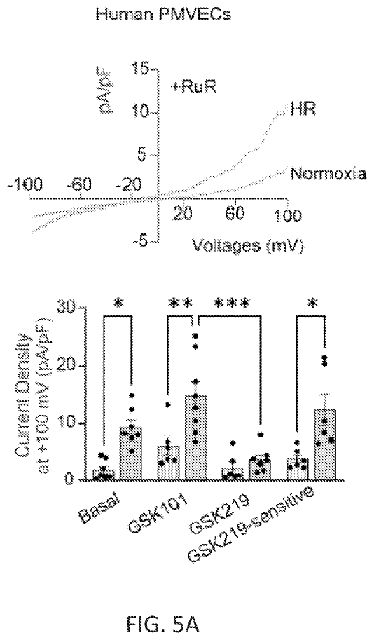

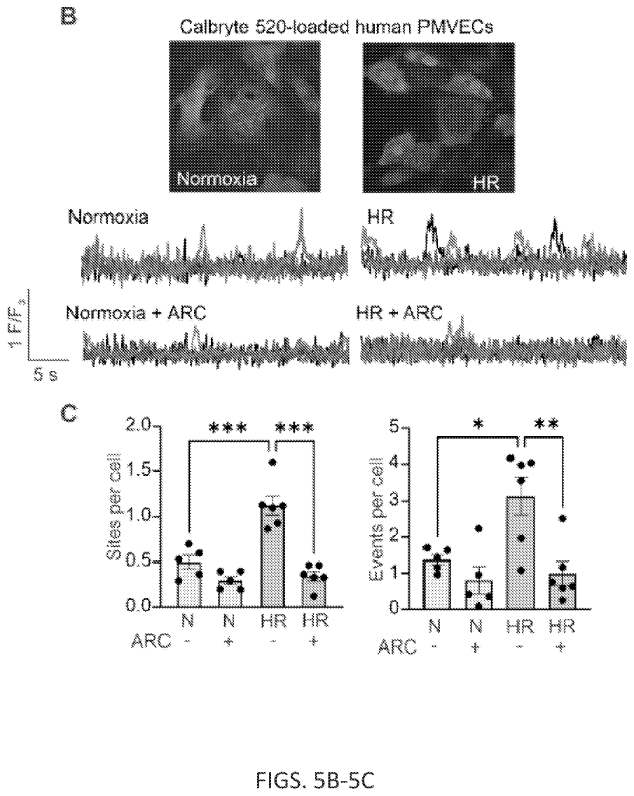

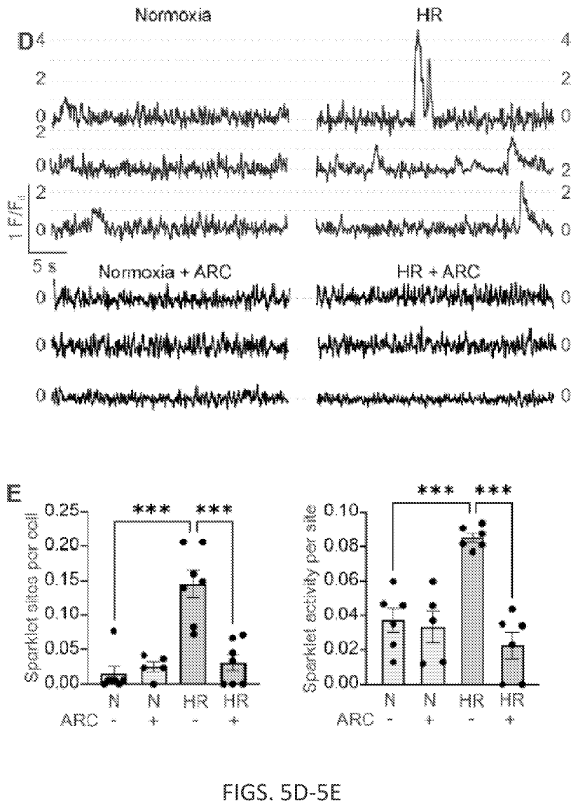

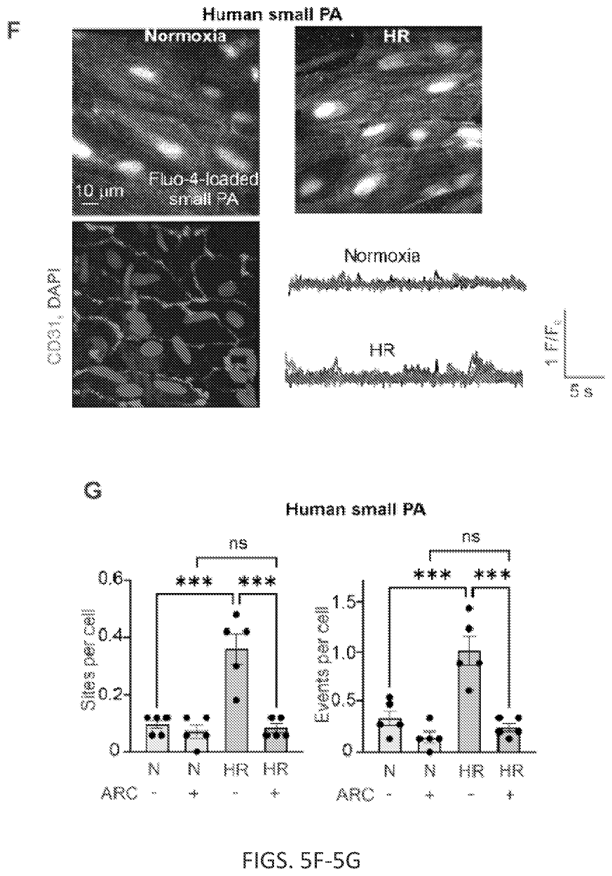

To determine whether acute HR-induced increases in TRPV4EC channel activity and endothelial Ca²⁺ signals are also observed in human pulmonary endothelium, we exposed human PMVECs to HR (1 hour hypoxia followed by 10 minutes reoxygenation) or to normoxia for 70 minutes, after which TRPV4EC channel activity and endothelial Ca²⁺ signals were measured. TRPV4 currents, defined as those sensitive to the TRPV4 inhibitor, GSK219 (100 nM), were increased in human PMVECs (FIG. 5A) by HR exposure, providing the first direct evidence that acute HR exposure increases TRPV4 channel activity in the human pulmonary endothelium.

To record individual Ca²⁺ signals in human PMVECs, we included the Ca²⁺ indicator, Calbryte520 (0.1 μM), during the last 10 minutes of incubation under reoxygenation or normoxia conditions. Baseline Ca²⁺ signaling (FIGS. 5B and 5C) and TRPV4EC sparklet activity (FIGS. 5D and 5E) were increased in human PMVECs after HR exposure. Similar to our findings in murine PMVECs. HR induced an increase in baseline endothelial Ca signaling as well as TRPV4EC sparklets in human PMVECs, an effect that was blocked by treatment with the selective P2Y2R inhibitor, ARC (FIG. 5E). These findings indicate that acute HR exposure induces TRPV4EC channel activity in a P2Y2R-dependent manner in both mouse and human PMVECs.

Freshly isolated small Pas (~50 μm) from human lungs also showed an increase in baseline endothelial Ca²⁺ signaling after HR exposure (FIGS. 5F and G). Moreover, HR-induced increase in endothelial Ca²⁺ signaling was inhibited by P2Y2R inhibitor ARC in human small Pas (FIG. 5G). These data suggested that HR exposure results in a P2Y2R-dependent increase in endothelial Ca²⁺ signaling in human small Pas.

eATP efflux through endothelial Panx1 channels mediates IR-induced activation of P2Y2REC-TRPV4EC signaling in the pulmonary endothelium.

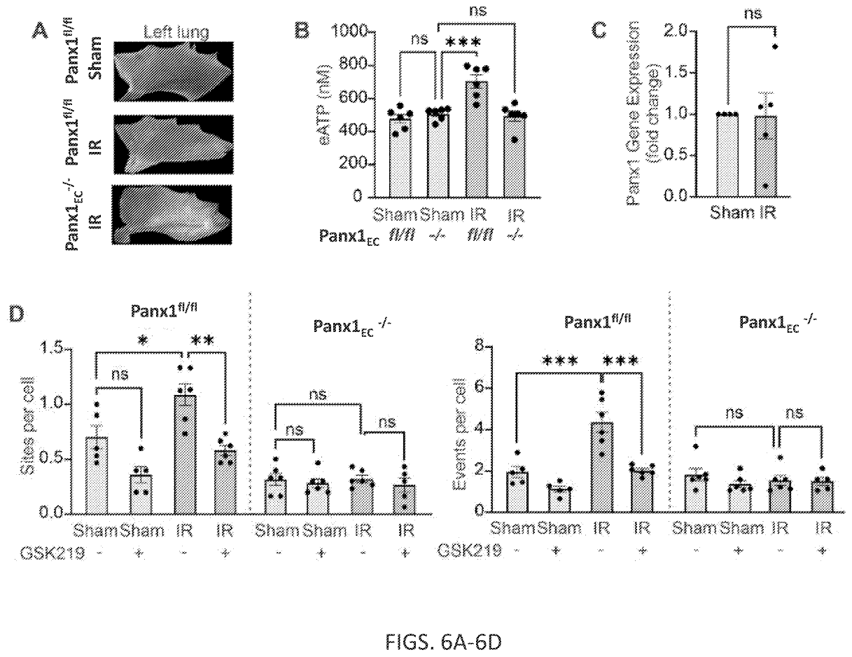

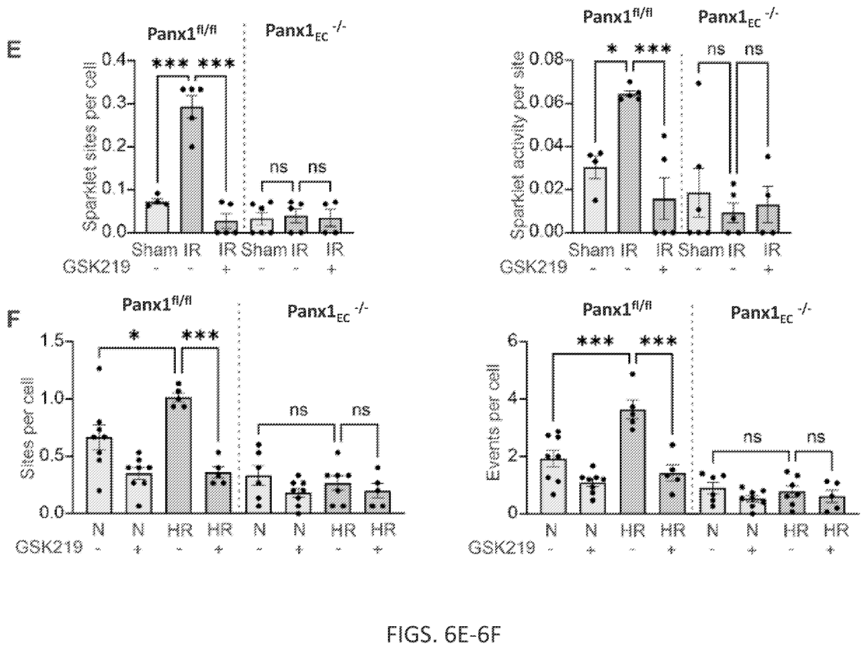

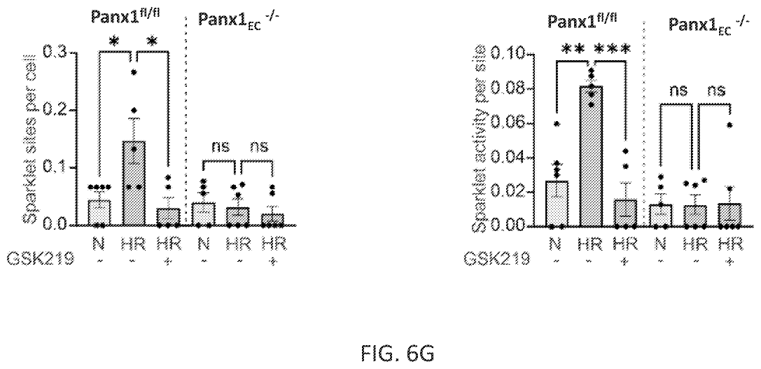

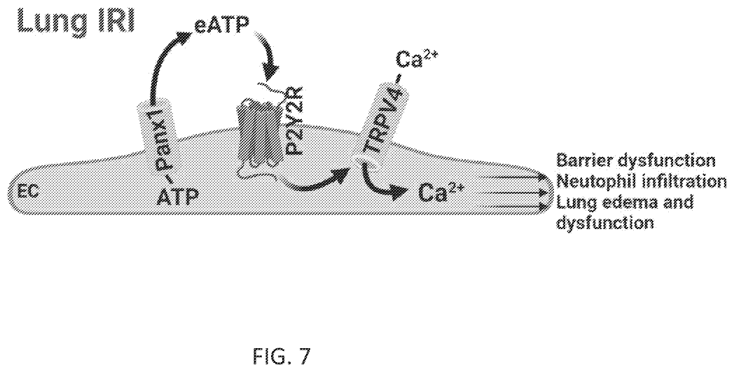

We have recently shown that activation of endothelial Panx1 channels increases eATP levels in small Pas under normal conditions.⁸ Moreover, lung IR increased eATP levels in BAL fluid through endothelial Panx1 activation.⁹ Consistent with these reports, we show that PanxIEC−/− mice are protected against IR-induced gross lung injury (FIG. 6A).

Bioluminescence measurements showed that lung IR induced eATP release from small Pas; this effect was absent in small Pas from Panx1EC−/− mice (FIG. 6B), suggesting that lung IR increases eATP efflux through endothelial Panx1 channels. Endothelial Panx1 mRNA expression was not altered after lung IR (FIG. 6C), suggesting that increased eATP efflux after lung IR is a result of increased activity, not expression, of endothelial Panx1 channels. To determine the effect of Panx1-mediated eATP efflux on endothelial Ca²⁺ signaling after lung IR. we recorded baseline endothelial Ca²⁺ signals and TRPV4 sparklet activity in small Pas from Panx1EC−/− and Panx1fl/fl mice after IR or sham surgery. As expected, the activity of baseline endothelial Ca²⁺ signaling (FIG. 6D) and TRPV4EC sparklets (FIG. 6E) was elevated in control Panx1fl/fl mice after lung IR compared with sham mice. However, no increase in baseline endothelial Ca²⁺ signaling or TRPV4EC sparklets was observed in the endothelium from Panx1EC−/− mice following IR (FIGS. 6D and 6E). Taken together, these data indicate that increased eATP efflux through endothelial Panx1 channels activates downstream P2Y2REC-TRPV4EC signaling after lung IR. In addition, HR-induced increases in the baseline activity of endothelial Ca²⁺ signals (FIG. 6F) and TRPV4EC sparklets (FIG. 6G) were also absent in ex vivo small Pas from Panx1EC−/− mice. Collectively, these findings support the concept that eATP effluxed via endothelial Panx1 channels activates endothelial P2Y2R-TRPV4 signaling, resulting in lung inflammation, endothelial barrier disruption, edema, and lung dysfunction after lung IR.

### DISCUSSION

Several previous studies provided evidence that endothelial TRPV4 channels are involved in various models of acute lung injury^{12,13,15}. We recently showed that endothelial TRPV4 channels play a role in lung IRI.¹⁶ Our current study provides novel insights into the purinergic mechanisms that mediate lung IR-induced activation of endothelial TRPV4 channels and subsequent lung injury. We show that lung IR increases endothelial Panx1 channel-mediated efflux of eATP, which then activates endothelial P2Y2R signaling. This, in turn, increases the activity of endothelial TRPV4 channels, resulting in endothelial barrier disruption, lung edema, immune cell infiltration, and lung dysfunction after IR. Activation of this endothelial Panx1-eATP-P2Y2R-TRPV4 signaling axis was observed in in vivo, ex vivo. and in vitro models of IR. Acute H R-induced P2Y2R-TRPV4 signaling was also observed in human PMVECs and endothelium from human small Pas. Overall, our data provide evidence for a novel role of endothelial P2Y2Rs in IR-induced lung edema and dysfunction and suggest that P2Y2Rs could be therapeutically targeted to prevent lung IRI, and subsequent primary graft dysfunction, after lung transplantation. The conclusions of this study may also assist in future investigations of the interplay between endothelial mechanisms underlying lung IRI with those from immune cells and epithelial cells.

eATP rapidly accumulates at sites of inflammation including IRI sites.⁹ The proinflammatory effects of eATP are largely attributable to P1 and P2 receptors at the cell membrane. These receptors are widely expressed in pulmonary endothelium, epithelium, and a variety of inflammatory cells.²⁶⁻²⁸ G protein-coupled P2Y receptors (P2Y1,2,3,6.11-14) and ligand-gated P2× ion channels (P2XI-7) make up the P2 receptor family.^{26,29} Among P2Y receptor subtypes, P2Y2R has been most commonly linked to inflammation and IRL.^{26,29} P2Y2R deletion was shown to be protective in several models of acute inflammation.²⁶ Moreover, endothelial P2Y2Rs were found to promote inflammatory cytokine production in atherosclerosis.³⁰ P2X receptors, especially the P2X7 receptor. have also been shown to play important roles in inflammatory disorders.²⁶ Our present study shows that the endothelial P2Y2R is involved in lung inflammation, edema, and dysfunction after lung IR.

When activated by various stress stimuli, Panx1 channels on the cell membrane are known to release ATP into the extracellular space.³¹ Upstream mechanisms involved in increasing the activity of endothelial Panx1 channels in lung IRI are not yet known. Panx1 channels can be activated by many stimuli, including, but not limited to, TNFα, mechanical stretch, membrane depolarization, a rise in intracellular Ca²⁺, and temperature.^{31,32} Notably, TNFα levels are increased in the lungs after IR, and we previously demonstrated that macrophage-dependent TNFα secretion is an early trigger for inflammation during lung IRI.^{33,34} Our previous studies on the pulmonary endothelium showed that, under physiological conditions, Ca²⁺ influx through endothelial TRPV4 channels does not activate Panx1.⁸ However, supraphysiological activation of TRPV4 channels with the agonist, GSK101 (10 nM), increased eATP efflux through Panx1 channels (FIG. 11). raising the interesting possibility that Panx1-P2Y2R-mediated stimulation of TRPV4 channels may lead to a feed-forward loop that activates Panx1, and in turn, results in further, rapid activation of TRPV4 channels.

Lung IRI involves a complex interplay between immune cells and pulmonary endothelial and epithelial cells.^{16,33,35} TRPV4 channels in epithelial cells and immune cells have been implicated in lung dysfunction and edema in lung injury.³⁶⁻³⁸ Panx1 expression has also been reported in epithelial and immune cells.³⁹⁻⁴⁰ Our demonstration that IR-induced lung edema and dysfunction are absent in Panx1EC−/−⁹, P2Y2REC−/−. and TRPV4EC−/− ¹⁶ mice suggests a central role for an endothelial cell Panx1-P2Y2R-TRPV4 signaling axis in lung IRI. Studies in epithelial-, macrophage-, and neutrophil-specific Panx1/P2Y2R/TRPV4-knockout mice will be required to identify a potential role for a Panx1-P2Y2R-TRPV4 signaling axis in these cell types during lung IRI. Our results show that acute HR also increased the activity of the Panx1-P2Y2R-TRPV4 signaling axis in isolated small Pas as well as primary PMVECs. Because isolated small Pas and PMVECs (as used in our study) cannot recruit immune cells and these preparations lack epithelial cells, we posit that HR- or IR-induced effects on endothelial Ca²⁺ signaling originate at the level of pulmonary microvascular Ecs.

The present study demonstrates that endothelial deletion of P2Y2R is sufficient to prevent IR-induced neutrophil infiltration. Functional P2Y2Rs are also expressed in neutrophils.⁴¹ However, the endothelial P2Y2R likely exerts indirect effects on neutrophil infiltration and function such that disruption of the pulmonary endothelial barrier by P2Y2R signaling increases neutrophil extravasation after IR. A similar mechanism was recently proposed to account for the beneficial effects of TRPV4 channel inhibition in a model of chemically induced lung injury.¹⁵ Our present study shows that IR-induced CXCL1, IL-6, and MCP-1 expression are significantly attenuated by endothelial P2Y2R deletion. We previously showed that endothelial deletion of TRPV4 attenuates IR-induced increases in several other inflammatory cytokines, including TNFα, IFNγ, and IL-17.⁵ These inflammatory cytokines are largely produced by immune cells in models of lung IRL.^{9,35} Our finding that endothelial deletion of P2Y2R reduces inflammatory cytokines in BAL fluid raises the interesting possibility that the endothelium may also be a source of inflammatory cytokines in lung IRI. Another possibility is that P2Y2R deletion from Ecs preserves endothelial barrier after IR, thereby potentially dampening overall inflammation and activation of innate immune cells. Although endothelial P2Y2Rs appear to be needed for the production of inflammatory mediators during lung IRI, whether other cell types also contribute to this effect is not clear.

The IRI component of primary graft dysfunction can be modeled in vivo using the hilar-ligation approach, a well-accepted model that has been used to simulate post-transplant IRL.⁴² We previously showed that this model induces inflammatory pathways common to experimental lung transplant models.⁹ Studies of individual Ca²⁺ signals in the intact endothelium of small Pas offers advantages over in vitro studies of EC monolayers or investigations of whole-cell increases in Ca²⁺. However, it is likely that most pathological events in lung IRI occur at the level of the pulmonary capillary endothelium or alveolo-capillary barrier in lung IRI. Since a reliable analysis of individual Ca²⁺ signals in the intact pulmonary capillary endothelium is not technically feasible, small Pas offer an appropriate alternative option for studying the effects of lung IR on endothelial Ca²⁺ signals. The small Pas used in the current study are ~50 μm in diameter, have a single smooth muscle cell layer surrounding the EC layer,²¹ and lie close to the capillary bed.

In conclusion, our studies provide the first evidence that eATP, effluxed through endothelial Panx1 channels, activates endothelial P2Y2Rs, which, in turn, increases Ca²⁺ influx through TRPV4EC channels to mediate endothelial barrier disruption, neutrophil infiltration, edema, and dysfunction after lung IR. Thus, this novel endothelial Panx1-P2Y2R-TRPV4 signaling axis may be a key pathway that could be targeted therapeutically to prevent primary graft dysfunction after lung transplantation.

### BIBLIOGRAPHY

1. Laubach V E, Sharma A K. Mechanisms of lung ischemia-reperfusion injury. Curr Opin Organ Transplant. 2016:21:246-252. Doi: 10.1097/MOT.0000000000000304
2. Weyker P D, Webb C A, Kiamanesh D, Flynn B C. Lung ischemia reperfusion injury: a bench-to-bedside review. Semin Cardiothorac Vasc Anesth. 2013:17:28-43. Doi: 10.1177/1089253212458329
3. Fiser S M, Tribble C G, Long S M, Kaza A K, Kern J A, Jones D R, Robbins M K, Kron I L. Ischemia-reperfusion injury after lung transplantation increases risk of late bronchiolitis obliterans syndrome. Ann Thorac Surg. 2002; 73:1041-1047: discussion 1047-1048. Doi: 10.1016/s0003-4975(01)03606-2
4. Bharat A. Kuo E, Steward N, Aloush A, Hachem R, Trulock E P. Patterson G A, Meyers B F. Mohanakumar T. Immunological link between primary graft dysfunction and chronic lung allograft rejection. Ann Thorac Surg. 2008; 86:189-195; discussion 196-187. Doi: 10.1016/j.athoracsur.2008.03.073
5. Haywood N, Ta H Q, Rotar E, Daneva Z, Sonkusare S K, Laubach V E. Role of the purinergic signaling network in lung ischemia-reperfusion injury. Curr Opin Organ Transplant. 2021; 26:250-257. Doi: 10.1097/MOT.0000000000000854
6. In: Liedtke W B, Heller S, eds. TRP Ion Channel Function in Sensory Transduction and Cellular Signaling Cascades. Boca Raton (FL); 2007.
7. Yang Y, Delalio L J, Best A K. Macal E, Milstein J. Donnelly I, Miller A M, McBride M, Shu X, Koval M, et al. Endothelial Pannexin 1 Channels Control Inflammation by Regulating Intracellular Calcium. J Immunol. 2020; 204:2995-3007. Doi: 10.4049/jimmunol.1901089
8. Daneva Z, Ottolini M, Chen Y L, Klimentova E, Kuppusamy M, Shah S A, Minshall R D, Seye C I. Laubach V E. Isakson B E, et al. Endothelial pannexin 1-TRPV4 channel signaling lowers pulmonary arterial pressure in mice. Elife. 2021; 10. Doi: 10.7554/eLife.67777
9. Sharma A K, Charles E J, Zhao Y, Narahari A K, Baderdinni P K, Good M E, Lorenz U M, Kron I L, Bayliss D A. Ravichandran K S, et al. Pannexin-1 channels on endothelial cells mediate vascular inflammation during lung ischemia-reperfusion injury. Am J Physiol Lung Cell Mol Physiol. 2018; 315:L301-L312. Doi: 10.1152/ajplung.00004.2018
10. Daneva Z, Marziano C, Ottolini M, Chen Y L, Baker™, Kuppusamy M, Zhang A, Ta H Q, Reagan C E, Mihalek A D, et al. Caveolar peroxynitrite formation impairs endothelial TRPV4 channels and elevates pulmonary arterial pressure in pulmonary hypertension. Proc Natl Acad Sci USA. 2021; 118. Doi: 10.1073/pnas.2023130118
11. Marziano C, Hong K, Cope E L, Kotlikoff M I, Isakson B E, Sonkusare S K. Nitric Oxide-Dependent Feedback Loop Regulates Transient Receptor Potential Vanilloid 4 (TRPV4) Channel Cooperativity and Endothelial Function in Small Pulmonary Arteries. J Am Heart Assoc. 2017; 6. Doi: 10.1161/JAHA.117.007157
12. Sonkusare S K, Laubach V E. Endothelial TRPV4 channels in lung edema and injury. Curr Top Membr. 2022; 89:43-62. Doi: 10.1016/bs.ctm.2022.07.001
13. Hamanaka K, Jian M Y, Townsley M I, King J A, Liedtke W, Weber D S, Eyal F G, Clapp M M. Parker J C. TRPV4 channels augment macrophage activation and ventilator-induced lung injury. Am J Physiol Lung Cell Mol Physiol. 2010; 299:L353-362. Doi: 10.1152/ajplung.00315.2009
14. Hamanaka K, Jian M Y, Weber D S, Alvarez D F, Townsley M I, Al-Mehdi A B, King J A. Liedtke W. Parker J C. TRPV4 initiates the acute calcium-dependent permeability increase during ventilator-induced lung injury in isolated mouse lungs. Am J Physiol Lung Cell Mol Physiol. 2007; 293:L923-932. Doi: 10.1152/ajplung.00221.2007
15. Balakrishna S, Song W, Achanta S, Doran S F, Liu B, Kaelberer M M, Yu Z, Sui A, Cheung M, Leishman E, et al. TRPV4 inhibition counteracts edema and inflammation and improves pulmonary function and oxygen saturation in chemically induced acute lung injury. Am J Physiol Lung Cell Mol Physiol. 2014; 307:L158-172. Doi: 10.1152/ajplung.00065.2014
16. Haywood N, Ta H Q, Zhang A, Charles E J, Rotar E, Noona St, Salmon M, Daneva Z, Sonkusare S K. Laubach V E. Endothelial Transient Receptor Potential Vanilloid 4 Channels Mediate Lung Ischemia-Reperfusion Injury. Ann Thorac Surg. 2022; 113:1256-1264. Doi: 10.1016/j.athoracsur.2021.04.052
17. Chen X, Qian S, Hoggatt A, Tang H, Hacker T A, Obukhov A G, Herring P B, Seye C I. Endothelial Cell-Specific Deletion of P2Y2 Receptor Promotes Plaque Stability in Atherosclerosis-Susceptible ApoE-Null Mice. Arterioscler Thromb Vasc Biol. 2017; 37:75-83. Doi: 10.1161/ATVBAHA.116.308561
18. Lohman A W, Leskov I L, Butcher J T, Johnstone S R, Stokes T A, Begandt D, DeLalio L J, Best A K. Penuela S, Leitinger N, et al. Pannexin 1 channels regulate leukocyte emigration through the venous endothelium during acute inflammation. Nat Commun. 2015:6:7965. Doi: 10.1038/ncomms8965
19. Ottolini M, Hong K, Cope E L, Daneva Z, DeLalio L J, Sokolowski J D, Marziano C, Nguyen N Y, Altschmied J, Haendeler J, et al. Local peroxynitrite impairs endothelial transient receptor potential vanilloid 4 channels and elevates blood pressure in obesity. Circulation. 2020:141:1318-1333. Doi: 10.1161/CIRCULATIONAHA.119.043385
20. Yang Z, Sharma A K, Marshall M, Kron I L, Laubach V E. NADPH oxidase in bone marrow-derived cells mediates pulmonary ischemia-reperfusion injury. Am J Respir Cell Mol Biol. 2009:40:375-381. Doi: 10.1165/rcmb.2008-03000C
21. Daneva Z, Chen Y L, Ta H Q, Manchikalapudi V, Bazaz A, Laubach V E, Sonkusare S K. Endothelial I K and SK channel activation decreases pulmonary arterial pressure and vascular remodeling in pulmonary hypertension. Pulm Circ. 2023; 13:e12186. Doi: 10.1002/pul2.12186
22. Ottolini M. Daneva Z, Chen Y L. Cope E L, Kasetti R B, Zode G S, Sonkusare S K. Mechanisms underlying selective coupling of endothelial Ca²⁺ signals with eNOS vs. IK/SK channels in systemic and pulmonary arteries. Journal of Physiology. 2020; 598:3577-3596. Doi: 10.1113/JP279570
23. Sonkusare S K, Bonev A D, Ledoux J, Liedtke W, Kotlikoff M I, Heppner T J, Hill-Eubanks D C, Nelson M T. Elementary Ca²⁺ signals through endothelial TRPV4 channels regulate vascular function. Science. 2012; 336:597-601. Doi: 10.1126/science.1216283
24. Chen Y L, Daneva Z, Kuppusamy M, Ottolini M, Baker™, Klimentova E, Shah S A, Sokolowski J D, Park M S, Sonkusare S K. Novel Smooth Muscle Ca(2+)-Signaling Nanodomains in Blood Pressure Regulation. Circulation. 2022; 146:548-564. Doi: 10.1161/CIRCULATIONAHA.121.058607
25. Ohanyan V, Raph S M, Dwenger M M, Hu X, Pucci T, Mack G, Moore J Bt, Chilian W M, Bhatnagar A, Nystoriak M A. Myocardial Blood Flow Control by Oxygen Sensing Vascular Kvbeta Proteins. Circ Res. 2021; 128:738-751. Doi: 10.1161/CIRCRESAHA. 120.317715
26. Linden J, Koch-Nolte F, Dahl G. Purine Release, Metabolism, and Signaling in the Inflammatory Response. Annu Rev Immunol. 2019; 37:325-347. Doi: 10.1146/annurev-immunol-051116-052406
27. Olotu C, Kiefmann M, Ronneburg C. Lehmensiek F, Cuvenhaus A. Meidl V, Goetz A E, Kiefmann R. Analysis of purine receptor expression and functionality in alveolar epithelial cells. Purinergic Signal. 2020; 16:213-229. Doi: 10.1007/s11302-020-09696-0
28. Le T T, Berg N K, Harting M T, Li X, Eltzschig H K, Yuan X. Purinergic Signaling in Pulmonary Inflammation. Front Immunol. 2019:10:1633. Doi: 10.3389/fimmu.2019.01633
29. Di Virgilio F, Sarti A C, Coutinho-Silva R. Purinergic signaling, DAMPs, and inflammation. Am J Physiol Cell Physiol. 2020; 318:C832-C835. Doi: 10.1152/ajpcell.00053.2020
30. Jin H. Ko Y S, Park S W, Kim H J. P2Y(2)R activation by ATP induces oxLDL-mediated inflammasome activation through modulation of mitochondrial damage in human endothelial cells. Free Radic Biol Med. 2019; 136:109-117. Doi: 10.1016/j.freeradbiomed.2019.04.004
31. Navis K E, Fan C Y, Trang T, Thompson R J, Derksen D J. Pannexin 1 Channels as a Therapeutic Target: Structure. Inhibition, and Outlook. ACS Chem Neurosci. 2020:11:2163-2172. Doi: 10.1021/acschemneuro.0c00333
32. Sanchez Arias J C, Wicki-Stordeur L E, Candlish R C, van der Slagt E, Paci I, Rao P P N, MacVicar B A, Swayne L A. PANX1 in inflammation heats up: New mechanistic insights with implications for injury and infection. Cell Calcium. 2020:90:102253. Doi: 10.1016/j.ceca.2020.102253
33. Sharma A K, Fernandez L G, Awad A S, Kron I L, Laubach V E. Proinflammatory response of alveolar epithelial cells is enhanced by alveolar macrophage-produced TNF-alpha during pulmonary ischemia-reperfusion injury. Am J Physiol Lung Cell Mol Physiol. 2007; 293:L105-113. Doi: 10.1152/ajplung.00470.2006
34. Zhao M, Fernandez L G, Doctor A, Sharma A K, Zarbock A, Tribble C G, Kron I L, Laubach V E. Alveolar macrophage activation is a key initiation signal for acute lung ischemia-reperfusion injury. Am J Physiol Lung Cell Mol Physiol. 2006; 291:L1018-1026. Doi: 10.1152/ajplung.00086.2006
35. Sharma A K, LaPar D J, Zhao Y, Li L, Lau C L, Kron I L, Iwakura Y, Okusa M D, Laubach V E. Natural killer T cell-derived IL-17 mediates lung ischemia-reperfusion injury. Am J Respir Crit Care Med. 2011; 183:1539-1549. Doi: 10.1164/rccm.201007-11730C
36. Weber J, Rajan S, Schremmer C, Chao Y K, Krasteva-Christ G, Kannler M, Yildirim A O, Brosien M. Schredelseker J, Weissmann N, et al. TRPV4 channels are essential for alveolar epithelial barrier function as protection from lung edema. JCI Insight. 2020; 5. Doi: 10.1172/jci.insight.134464
37. Scheraga R G, Southern B D, Grove L M. Olman M A. The Role of Transient Receptor Potential Vanilloid 4 in Pulmonary Inflammatory Diseases. Front Immunol. 2017; 8:503. Doi: 10.3389/fimmu.2017.00503
38. Scheraga R G, Southern B D, Grove L M, Olman M A. The Role of TRPV4 in Regulating Innate Immune Cell Function in Lung Inflammation. Front Immunol. 2020; 11:1211. Doi: 10.3389/fimmu.2020.01211
39. Chen W, Zhu S, Wang Y, Li J, Qiang X, Zhao X, Yang H, D'Angelo J, Becker L, Wang P, et al. Enhanced Macrophage Pannexin 1 Expression and Hemichannel Activation Exacerbates Lethal Experimental Sepsis. Sci Rep. 2019; 9:160. Doi: 10.1038/s41598-018-37232-z
40. Krick S, Wang J, St-Pierre M, Gonzalez C, Dahl G, Salathe M. Dual Oxidase 2 (Duox2) Regulates Pannexin 1-mediated ATP Release in Primary Human Airway Epithelial Cells via Changes in Intracellular pH and Not H₂O₂ Production. J Biol Chem. 2016; 291:6423-6432. Doi: 10.1074/jbc.M115.664854
41. Chen Y. Corriden R, Inoue Y, Yip L. Hashiguchi N, Zinkernagel A, Nizet V, Insel P A, Junger W G. ATP release guides neutrophil chemotaxis via P2Y2 and A3 receptors. Science. 2006; 314:1792-1795. Doi: 10.1126/science.1132559
42. Lama V N, Belperio J A, Christie J D, El-Chemaly S, Fishbein M C, Gelman A E, Hancock W W, Keshavjee S. Kreisel D. Laubach V E. et al. Models of Lung Transplant Research: a consensus statement from the National Heart, Lung, and Blood Institute workshop. JCI Insight. 2017; 2. Doi: 10.1172/jci.insight.93121

### Example 2

### Introduction

Ischemia-reperfusion injury (IRI) after lung transplantation is a major cause of primary graft dysfunction (PGD), a feared peri-operative complication occurring in up to 30% of recipients that is associated with deleterious short and long-term outcomes.¹⁻³ Patients with PGD have nearly seven times greater risk of all-cause operative mortality at 30 days post-transplant and are more likely to experience chronic lung allograft dysfunction.^{4,5} The incidence of PGD continues to rise, further emphasizing the need for novel therapeutics.⁶

Efforts to mitigate IRI, and thereby reduce PGD rates, after lung transplantation are ongoing. Recent studies indicate that transient receptor potential vanilloid 4 (TRPV4) channels are an important mediator of IRI, and that both pharmacologic TRPV4 antagonism as well as TRPV4 gene knockout attenuates lung IRI⁷⁻¹⁰ TRPV4 is a cation channel expressed in multiple cell types including epithelial cells, immune cells, and vascular endothelium.¹ Activation of TRPV4 channels has been linked to lung injury through endothelial barrier disruption, edema, and inflammatory cell recruitment and activation.¹²⁻¹⁴ Our laboratory demonstrated that endothelial TRPV4 knockout mice, as well as wild-type mice pretreated with GSK2193874 (GSK219), a selective TRPV4 inhibitor, had significant attenuation of lung IRI using a left hilar ligation model.^{7,15} We have also identified a signaling axis leading to TRPV4 activation in endothelial cells whereby pannexin-1 (Panx1) channel-mediated efflux of adenosine triphosphate (ATP) activates TRPV4 via P2Y2 receptor (P2Y2R) activation, leading to endothelial barrier disruption and lung IRI.¹⁵ However, a limitation of these studies was that the murine hilar ligation model does not entail transplantation. Therefore, the purpose of the current study was to examine the effect of TRPV4 inhibition on allograft function using a clinically relevant porcine transplantation model of lung IRI. We hypothesized that recipient animals treated with GSK219 would demonstrate significant attenuation of lung IRI in a porcine transplant model.

### Materials and Methods

### Animals and Study Design

The University of Virginia's Animal Care and Use Committee approved this study, protocol 4262 (approved 10/23/2021). All procedures were performed in compliance with the National Institutes of Health 1996 Guide for the Care and Use of Laboratory Animals. Ten farm-raised, domestic swine of both sexes (31.6-60.0 kg weight range) were utilized. Animals were randomized to one of two groups (n=5/group). A treatment group received 1 mg/kg GSK219 (MedChemExpress, Monmouth Junction, NJ), a potent and selective TRPV4 inhibitor^{16,17} that we have previously demonstrated to attenuate lung IRI in mice pretreated with a 1 mg/kg intravenous dose.⁷ A control group received vehicle only (2% DMSO in 0.9% sodium chloride containing 20 mg/ml Kolliphor H S15, Sigma-Aldrich, St. Louis, MO). GSK219 or vehicle was administered as a systemic, intravenous infusion (100 ml volume over a 30-min period via central venous line placed in the left external jugular vein) beginning after the recipient animal's left pulmonary artery (PA) was clamped (i.e. at the time of recipient lung explant; approximately 40 min prior to initiation of graft reperfusion). All infusions were completed prior to reperfusion of the implanted lung. All surgeries and treatments were performed in a blinded fashion where the treatment identities were not revealed until after the study was completed.

### Donor and Procurement Procedure

A porcine left lung transplant model was utilized as previously described by our laboratory¹⁸, with modifications as described here. On day 1, the donor animal was sedated and intubated. General anesthesia was maintained while animals were ventilated with 100% oxygen to measure baseline parameters. Lungs were ventilated to a tidal volume of 8.0 mL/kg with a respiratory rate of 22 breaths/min and a positive end-expiratory pressure of 5.0 cm H₂O. Intravenous heparin (200 IU/kg, APP Pharmaceuticals, Schaumburg, IL) was administered.

Donor animals underwent a median sternotomy and pericardiotomy to expose the heart, great vessels, and lungs. Prostaglandin E1 (10 mg/kg, Pfizer, New York, NY) was injected into the main PA. Cardiac death was initiated by ligation of the superior and inferior vena cava and venting of the left atrium. The lungs were then flushed with 1 L room temperature heparinized Perfadex (XVIVO Perfusion. Inc, Englewood, CO; 10,000 IU heparin per liter) via the cardioplegia cannula. At this point the lungs underwent 30 min of warm ischemia. At the conclusion of warm ischemia, the thoracic cavity was filled with cold (4° C.) slush, and lungs were flushed with an additional 1 L cold Perfadex via the main PA. At mid-inspiration, the trachea was clamped, and the heart-lung bloc was explanted. The lungs were then flushed retrograde with an additional IL cold Perfadex and placed in 4° C. cold storage for 24 hrs.

### Lung Transplantation and Reperfusion

On day 2, recipient animals were anesthetized and ventilated as above for donor animals. A left thoracotomy was performed. and the left lung hilum was exposed. Central arterial and venous access was obtained for monitoring. Left pneumonectomy and left lung transplantation were performed as previously described.¹⁸ Lung function was monitored during 4 hours of reperfusion while the left thoracotomy remained open. Combined (left and right) lung compliance was monitored throughout reperfusion. Airway pressure measurements and partial pressure of arterial oxygen (PaO₂; systemic arterial as well as direct left pulmonary vein samples for graft-specific oxygenation) were obtained after 15 minutes, 1 hour and hourly thereafter. The average PaO₂ of the left superior and left inferior pulmonary veins were reported. PaO₂, PaCO₂, and pH were measured using an i-STAT handheld blood analyzer (Abbott Point of Care, Princeton, NJ). After 4 hours of reperfusion, the animal was euthanized, and the heart-lung bloc was excised.

### Lung Sampling

After completion of reperfusion, small peripheral samples of the transplanted left lungs were tied off, and fresh tissue samples were collected from the left upper lobe, the left lingula, and left lower lobe. The same surgeon collected all samples from consistent, peripheral, dependent locations of each lobe in all animals. These samples were either used fresh or flash-frozen in liquid nitrogen and stored at −80° C.

### Lung Wet/Dry Weight

Fresh lung samples were patted dry, weighed, and dried in a vacuum oven at 56° C. until the weight stabilized (typically 3-4 days). Lung wet/dry weight ratios were then calculated to assess edema.

### Bronchoalveolar Lavage (BAL) and Differential Cell Counts

After collection of fresh lung samples, the left upper lobe was lavaged using 40 ml normal saline. Total cell counts and differential cell counts in BAL fluid were performed by the University of Virginia Clinical Laboratory in a blinded fashion. Total cell counts were performed using a hemocytometer. For differential cell counts, slides were prepared and stained using a Wescor Aerospray Hematology Slide Stainer/Cytocentrifuge (ELITechGroup Inc., Logan, UT), and differential cells (macrophages, neutrophils, lymphocytes, and eosinophils) were counted manually on a microscope. For each slide, 5 random microscopic fields at 40× magnification were evaluated, and the counts were averaged and expressed as a percentage of the total cells counted. Additional aliquots of BAL fluid were centrifuged (400 RCF, 10 minutes. 4° C.), and the supernatants were stored at −80° C.

### Histopathology and Lung Injury Scoring

After obtaining all fresh tissue samples, the left lower lobe was inflated with 10% buffered formalin (by gravity to a pressure of 25 cm H₂O) through the trachea. The trachea was clamped, and the lobe was submerged in formalin and fixed for 36 hours at 4′C. Fixed, peripheral samples from two predefined locations per lobe were collected, immersed in 70% ethanol at 4° C. for 2-3 days, and paraffin embedded. Lung sections were stained with hematoxylin-eosin and evaluated by a lung pathologist, blinded to group assignment, to assess lung injury. Sections were scored using a standard scale as previously described¹⁹: alveolar edema (0≤5%, 1=625%, 2=2650%, 3>50%), neutrophils per high-powered field (0≤5, 1=6-10, 2=11-20, 3>20), interstitial inflammation (0=none, 1=minimal, 2=moderate, 3=severe), composite score=sum of all scores (range, 0-9). The histology scores from the two lung sections were then averaged to yield a single score per animal for each parameter.

### Neutrophil Immunostaining and Counting

Neutrophils were identified in lung sections by immunohistochemistry using a rat anti-mouse neutrophil antibody (LY6B.2: Abcam. Waltham. MA). Alkaline phosphatase-conjugated anti-rat immunoglobulin G (Sigma-Aldrich) was used as a secondary antibody, and immune signals were detected using Fast-Red (Sigma-Aldrich). Normal rat immunoglobulin G (eBioscience, San Diego, CA) was used as a negative control. Sections were counterstained with hematoxylin. For each lung section. neutrophils were counted, in a blinded fashion, in five random fields at 20× magnification and averaged. These cell counts did not distinguish between cells in various compartments of the lung (airspace, interstitial, or marginated) and included all cells in peripheral lung tissue.

### Measurement of Adhesion Molecules, Junction Proteins, and Inflammatory Markers

Concentrations of interleukin-8 (IL-8), interleukin-Ibeta (IL-1β), soluble cell adhesion molecules (P-selectin, VCAM1, ICAM-1), soluble tight junction proteins (occludin, claudin-5, ZO-1, ZO-2), high mobility group box 1 (HMGB1), and angiopoietin-2 (Ang-2) were quantified in plasma or BAL fluid using ELISA kits (MyBioSource, San Diego, CA) according to the manufacturer's protocol.

### Statistical Analysis

Comparison of continuous values between groups was performed using ordinary one-way analysis of variance with post hoc Tukey's multiple comparison test. Continuous values are expressed as the mean±standard deviation, and analyses were performed using SAS 9.4 software (SAS Institute, Inc, Cary, NC). Significance between two groups was determined using Student t test and Prism 9 software (GraphPad Inc., La Jolla, CA). Data are presented using box-and-whisker plots where the lower and upper borders of the box represent the lower and upper quartiles (25^{th} percentile and 75^{th} percentile); the middle horizontal line represents the median; and the lower and upper whiskers represent the minimum and maximum data values. All data for each group are represented as individual symbols on each box-and-whisker plot. P<0.05 was used as the threshold for significance.

### Results

### Animals

There were no significant differences between the two groups of donor animals regarding systemic arterial PaO₂/FiO₂ ratio (490±16.4 vs. 502±13.1 mmHg, p=0.572) and dynamic lung compliance (19.5±2.02 vs. 18.2±2.29 mL/cm H₂O, p=0.691). All ten recipient animals survived the transplant and 4 hours of reperfusion without incident, and no adverse effects of GSK219 administration was observed.

### Lung Function

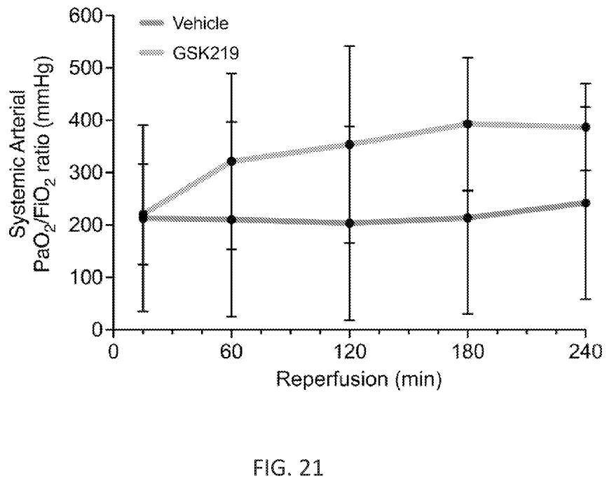

After transplantation, via direct left pulmonary vein sampling. oxygenation capacity of the transplanted lung was similar between groups at 15 minutes. 1 hour, and 2 hours of reperfusion. However, left lung-specific PaO₂/FiO₂ ratios were significantly improved in the TRPV4 inhibitor group at 3 hours (276±57 vs. 85.4±17.2 mmHg, P=0.026) and 4 hours (303±37 vs. 104±28.6 mmHg. P=0.003) of reperfusion versus vehicle control (FIG. 14A). Throughout reperfusion, there were no significant differences in pulmonary venous PaCO₂ or pH between vehicle-versus GSK-treated groups (average PaCO₂=60.1±2.11 vs. 60.4±1.46 mmHg; average pH=7.3±0.024 vs. 7.3±0.020). Systemic arterial PaO₂/FiO₂ ratios were improved during reperfusion in the GSK-treated group, although this was not significant (FIG. 21). Dynamic lung compliance was similar between groups throughout reperfusion (FIG. 14B).

### Lung Wet/Dry Weight

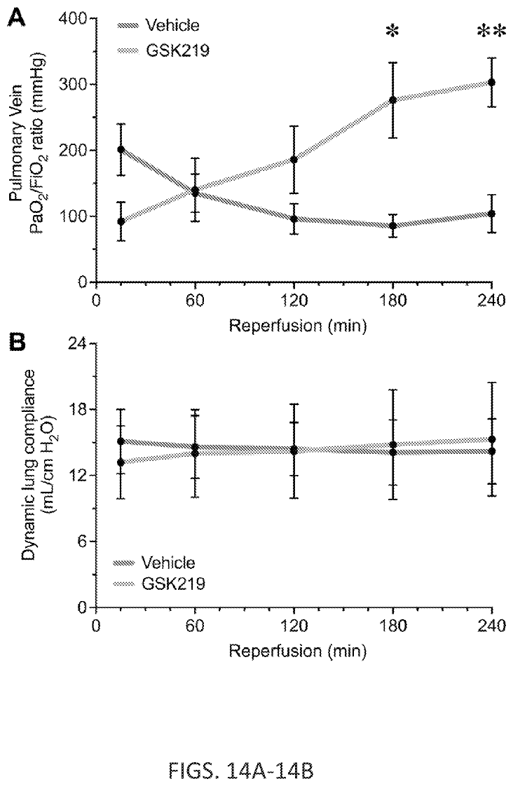

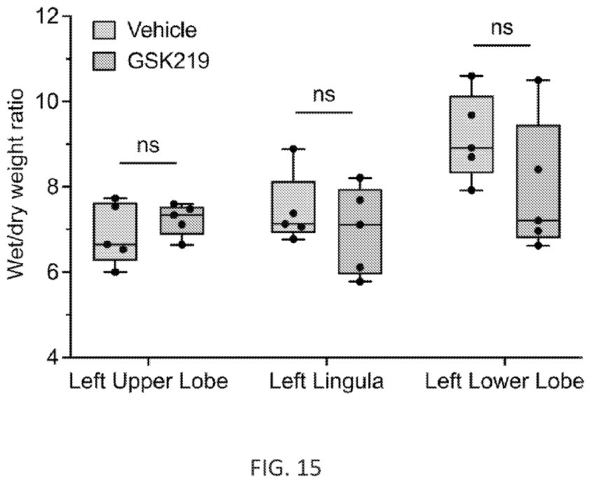

There were no significant differences in left lung wet/dry weight ratios between GSK219- and vehicle-treated groups following 4 hours of reperfusion (FIG. 15). However. wet/dry weight ratios of the left lingula and left lower lobe did trend lower in the TRPV4 inhibitor group.

### Lung Histopathology

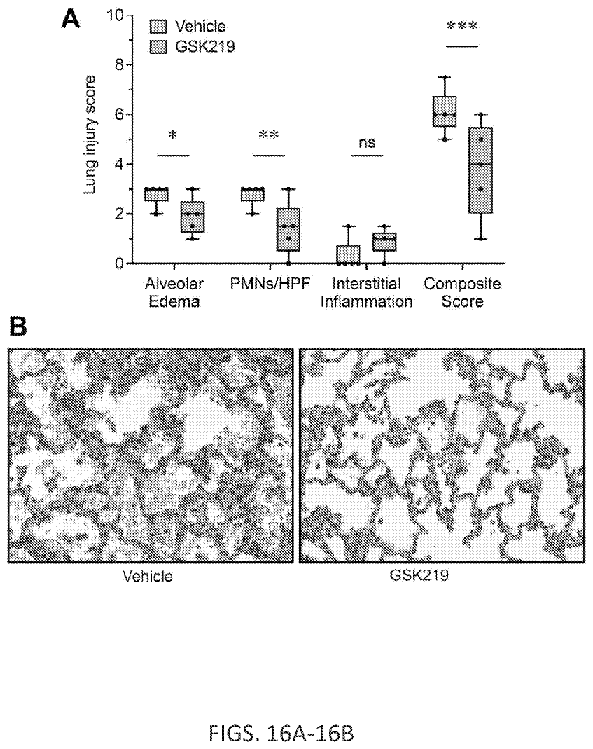

Histologic evaluation of lung sections revealed that scores for alveolar edema (P=0.048), neutrophils per high-powered field (P=0.028). and composite scores (P=0.041) were significantly reduced in the left lower lobes of the TRPV4 inhibitor group versus vehicle control (FIG. 16A). Overall, lung histology by H&E staining was significantly improved in the TRPV4 inhibitor group (FIG. 16B).

### Neutrophil Infiltration

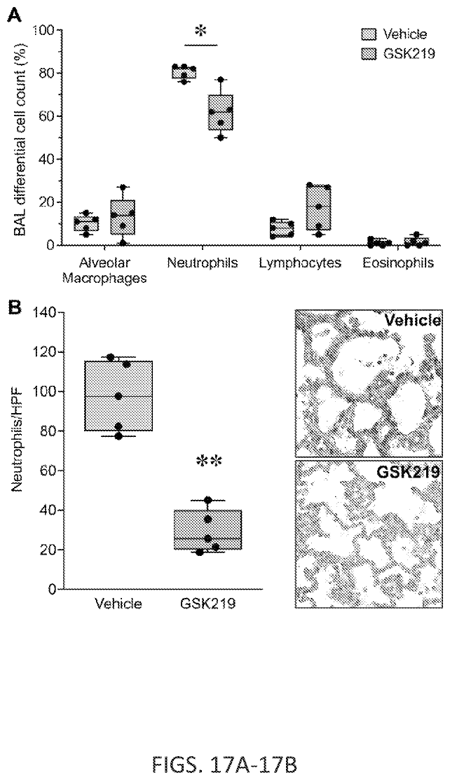

Differential cell counts of BAL fluid revealed significantly fewer neutrophils (percent of total cells) in the TRPV4 inhibitor group compared to vehicle control (61.8±4.4% vs. 80.2±1.4%, P=0.004) (FIG. 17A). Percentages of alveolar macrophages, lymphocytes, and eosinophils were similar between groups. Neutrophil infiltration, as measured by immunohistochemistry of lung sections, was significantly reduced by 70% in the TRPV4 inhibitor group compared to vehicle control (FIG. 17B, P<0.0001).

### Expression of Endothelial Cell Adhesion Molecules

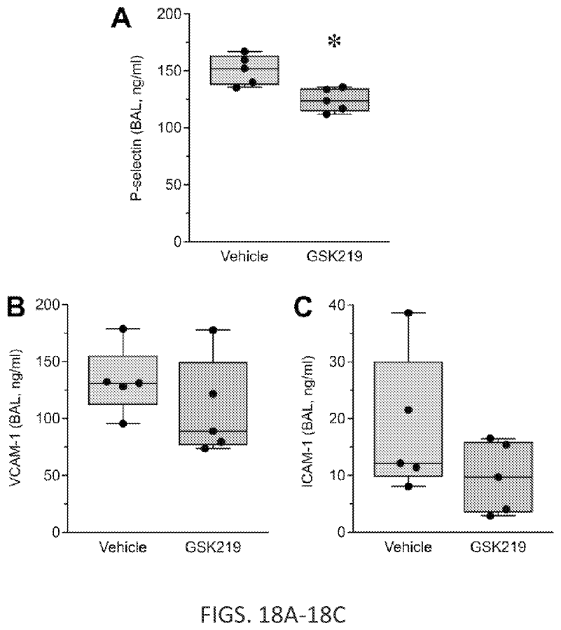

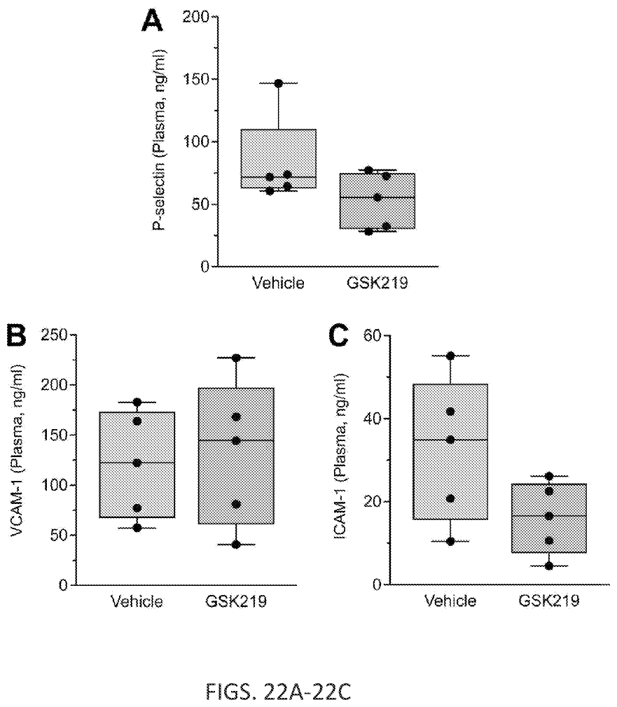

Following reperfusion, expression of soluble P-selectin in BAL fluid was significantly reduced in the TRPV4 inhibitor group versus vehicle control (FIG. 18A). Expression of soluble VCAM-1 and ICAM-1 in BAL fluid were also reduced in the TRPV4 inhibitor group, although this was not significant (FIGS. 18B-18C). Concentrations of soluble P-selectin, VCAM-1, and ICAM-1 in plasma were not significantly different between groups (FIGS. 22A-22C); however. P-selectin and ICAM-1 trended lower in the TRPV4 inhibitor group.

### Tight Junction Proteins

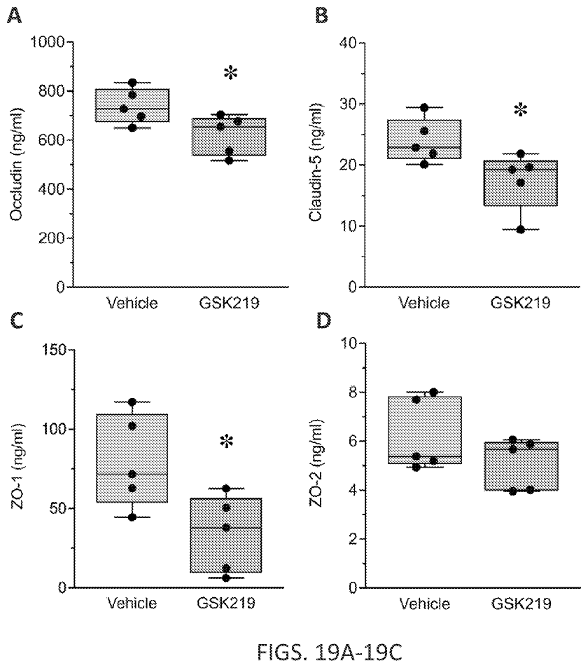

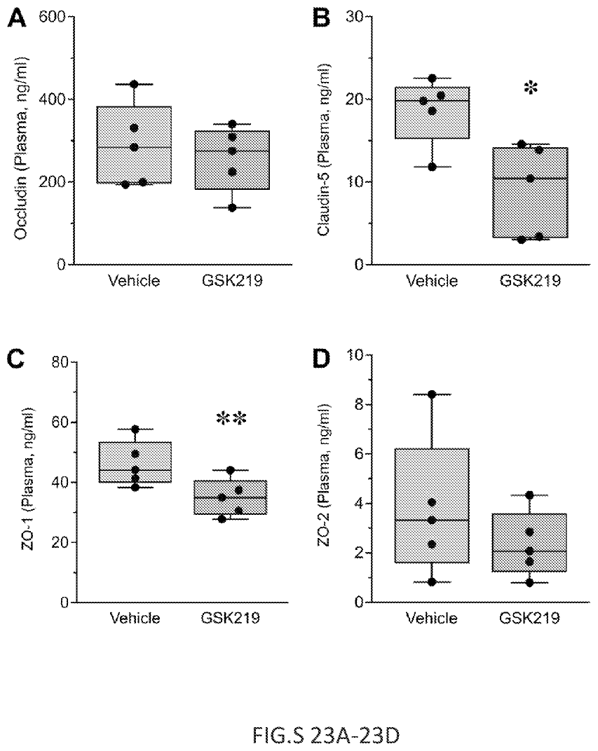

The concentration of the tight junction proteins occludin, claudin-5, and zonula occludens-1 (ZO-1) in BAL fluid were significantly reduced in the TRPV4 inhibitor group versus vehicle control (FIGS. 19A-19C). ZO-2 levels in BAL fluid were also reduced, but this was not statistically significant (FIG. 19D). Plasma concentrations of claudin-5 and ZO-1 were also significantly reduced in the TRPV4 inhibitor group (FIGS. 23B-23C).

### Inflammatory Biomarkers

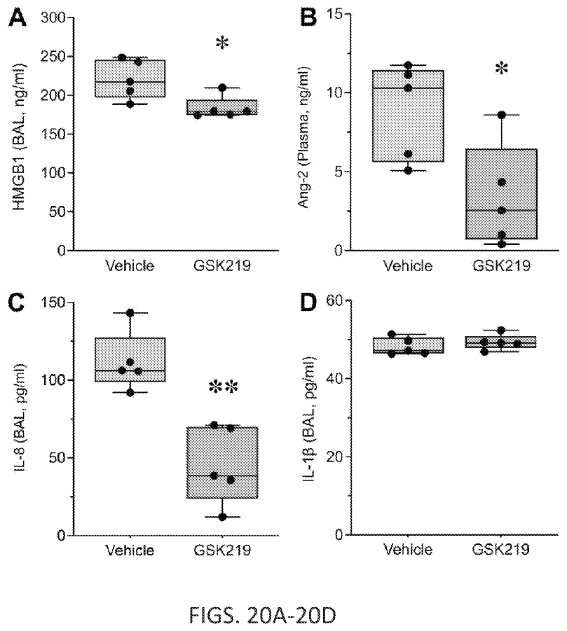

Concentrations ofHMGBT (FIG. 20A) and Ang-2 (FIG. 20B) in BAL fluid and plasma, respectively, were significantly reduced in the TRPV4 inhibitor group versus vehicle control. IL-8 levels in BAL fluid were significantly reduced in GSK219-treated animals (FIG. 20C); however. IL-1β levels were not significantly altered (FIG. 20D).

### DISCUSSION

We provide evidence that treatment of recipient animals with GSK219, a selective TRPV4 inhibitor, significantly attenuates lung IRI in a porcine transplant model. Importantly, post-transplant lung function was significantly improved in GSK219-treated animals as indicated by improved left pulmonary vein oxygenation at 3 and 4 hours of reperfusion. Improved left lung function was also reflected systemically where systemic arterial oxygenation was improved during reperfusion in the GSK-treated group, although this did not reach significance; perhaps due to variability in left versus right lung contributions. Here, we postulate that systemic oxygenation in the vehicle-treated group remains relatively flat during reperfusion because the left lung graft only gets a little worse over time, whereas in the GSK-treated animals, systemic oxygenation improved during reperfusion as the left lung graft significantly improved over time. This may suggest that GSK219 is not injuring the right lung, which is not expected.

Although recipient animals treated with GSK219 demonstrated reduced lung wet/dry weight in the left lower lobe, this difference did not reach statistical significance. However, histologic lung injury scoring demonstrated significantly reduced alveolar edema as well as reduced neutrophil infiltration and composite score in GSK219-treated animals. Attenuation of neutrophil infiltration by GSK219 treatment was confirmed by differential cell counts in BAL fluid and immunohistochemistry of lung sections. As supported by prior murine studies^{7,15,20} these results suggest that TRPV4 inhibition by GSK219 attenuated lung IRI, at least in part, via preservation of endothelial barrier integrity and reduced neutrophil infiltration.

Our group recently identified a purinergic mechanism of action for TRPV4 activation in endothelial cells after IR, where lung IR induces endothelial Panx1 channel-mediated efflux of ATP, which then activates endothelial P2Y2R signaling.¹⁵ In turn, this activates endothelial TRPV4 channels and calcium influx, resulting in endothelial barrier disruption, lung edema, neutrophil infiltration, and lung dysfunction after IR. Activation of this endothelial Panx1-P2Y2R-TRPV4 signaling axis was observed using in vivo, ex vivo, and in vitro models of IR.¹⁵ As such, we demonstrated that selective inhibition of Panx1²⁰, P2Y2R¹⁵, or TRPV4⁷ results in significant attenuation of lung dysfunction, endothelial permeability, edema, and inflammation after IR.

Our current study focused on evaluating biomarkers of endothelial barrier disruption since TRPV4 channels mediate endothelial barrier disruption during lung IRI in mice.^{7,15} Injury and inflammation involves endothelial cell activation, leading to expression of cell adhesion molecules, whose soluble forms can be detected in blood or BAL fluid²¹. Elevated expression of cell adhesion molecules leads to rolling, activation, and leukocyte adhesion to the endothelium, which is an important contributor to the pathogenesis of IRI.²²⁻²⁴ In the current study, soluble P-selectin was significantly reduced in BAL fluid of GSK219-treated animals. P-selectin, expressed by activated endothelial cells and platelets, has been referred to as the most important selectin for mediating leukocyte adhesion to vascular endothelium, and the use of soluble adhesion molecules as diagnostic markers of endothelial injury have become routinely utilized.²⁵

Tight junction proteins are critical for maintenance of endothelial and epithelial barriers. These barriers protect against potentially harmful molecules and excessive immune activation and are thus crucial for overall health. During injury or inflammation such as respiratory disease²⁶, tight junction proteins such as claudin-5, occludin, ZO-1, and ZO-2 are degraded and released from endothelium and consequently become detectable in blood²⁷⁻²⁹ or other fluids such as cerebrospinal fluid³⁰ to serve as biomarkers of barrier disruption. Thus, tight junction proteins were measured as biomarkers of endothelial barrier disruption after lung transplantation and reperfusion. In the present study, concentrations of several key endothelial tight junction proteins (occludin, claudin-5, ZO-1) in BAL fluid were significantly reduced in GSK219-treated animals, indicative of reduced degradation of tight junctions after transplant. This GSK219-mediated bolstering of tight junctions, and thus endothelial barrier integrity, likely contributes to the observed attenuation of neutrophil infiltration. Because tight junction proteins also play an important role in epithelial barrier integrity, we cannot exclude the possibility that TRPV4 inhibition may also preserve alveolar epithelial barrier integrity.

Ang-2, an endothelial growth factor that regulates vascular permeability, is a key mediator of acute lung injury and serves as a circulating biomarker of endothelial activation or injury.³¹ In the present study, improved endothelial barrier integrity by GSK219 treatment was evidenced by reduced expression of circulating Ang-2. In addition, expression of two key pro-inflammatory cytokines (IL-8 and IL-10 were measured, which we have previously shown to be elevated in BAL fluid in a porcine model of lung IRI after transplant.¹⁸ In the current study, expression of IL-8 in BAL fluid was significantly reduced in GSK219-treated animals, which helps explain the reduced neutrophil infiltration observed in these animals. IL-8, a potent neutrophil chemotactic factor, is expressed by various cell types including neutrophils, epithelial cells, alveolar macrophages, and endothelial cells. We previously demonstrated that exposure of murine pulmonary microvascular endothelial cells to acute hypoxia-reoxygenation induces secretion of CXCL1/KC, a murine IL-8 homologue, which was attenuated by GSK219.⁷ Additionally, elevated expression of CXCL1/KC in wild-type mice after lung IRI was attenuated in endothelial TRPV4 knockout mice.⁷ These data suggest that inhibition of TRPV4 channels by GSK219 attenuated porcine lung IRI after transplant, in part, by attenuating endothelial cell activation. Expression of IL-1 which is predominantly expressed by innate immune cells, was not altered by GSK219 treatment.

HMGB1. a damage-associated molecular pattern (DAMP) molecule released by necrotic or damaged somatic and immune cells, is an important facilitator of acute lung injury and numerous other lung pathologies³². Our laboratory has shown that HMGB1 mediates lung IRI and accumulates in BAL fluid after IR.³³ In the current study, HMGB1 levels were significantly reduced in BAL fluid of GSK219-treated animals. HMGB1 is known to induce endothelial permeability, and Wolfson et al. demonstrated that HMGB1 induces endothelial paracellular gap formation in concert with dissociation of intercellular junction proteins; key phenotypic changes leading to elevated endothelial permeability.³⁴ Thus, GSK219-mediated attenuation of HMGB1 production (among other biomarkers of barrier disruption and vascular inflammation discussed above) suggests that a principal mechanism for GSK219-mediated protection from post-transplant lung IRI involves preservation of endothelial barrier function and attenuation of neutrophil infiltration.

Although the protective effects of TRPV4 inhibition in acute injury are well documented, potential detrimental effects of TRPV4 inhibition needs mention. Most studies thus far have reported no adverse side effects after TRPV4 inhibitor treatment. Thomeloe et al. reported no effect on mean arterial pressure or heart rate in rats after daily, oral doses of up to 30 mg/kg GSK2193874 for 14 days.¹⁷ Goyal et al. reported that the TRPV4 inhibitor GSK2798745 was well tolerated in healthy volunteers and patients with stable heart failure.³⁵ We have shown that inhibition of TRPV4 channels in small pulmonary arteries ex vivo may result in a small, acute increase in pulmonary arterial pressure.³⁶ Because we have shown that TRPV4 inhibition by GSK219 is reversible³⁷, we would not expect that this effect would persist after acute treatment. Cheung et al. reported that GSK219 has a plasma half-life (t1/2) of 10 hours in rats and 31 hours in dogs.¹⁶ In the setting of lung transplant, early, acute treatment with TRPV4 inhibitor will be key for preventing PGD, and we would not anticipate significant or long-lasting adverse effects.

The current study builds upon our prior murine studies that utilized a left hilar ligation model where lung IRI is attenuated in endothelial TRPV4 knockout mice and in wild-type mice pretreated with GSK219.⁷¹⁵ The current study advances these results to a clinically relevant, large animal model of IRI after lung transplantation whereby treatment of recipient animals with a selective TRPV4 inhibitor significantly improves graft function and histology as well as reducing neutrophil infiltration. Reductions in key biomarkers of vascular inflammation and endothelial barrier disruption by GSK219 treatment suggest that a primary mechanism for improved lung function involves preservation of the microvascular endothelial barrier resulting in improved gas exchange and thus improved oxygenation. We propose that these effects are due largely to inhibition of TRPV4 channels on vascular endothelium, although inhibition of TRPV4 channels on other cell types could also be involved. For example, TRPV4 channels can facilitate activation of alveolar epithelial cells.” Mechanical ventilation can exert pathological forces on lung epithelial cells, which could potentially impact IRI, and TRPV4 inhibition was shown to improve the negative pathological stretch-response in lung cells during mechanical ventilation.³⁸ Thus, GSK219-mediated attenuation of lung IRI could also involve epithelial cells in addition to endothelial cells. In conclusion, our results suggest that pharmacologic inhibition of TRPV4 channels, which attenuates lung IRI in a porcine transplantation model, may be a valuable therapeutic strategy in the human lung transplant setting to prevent PGD.

### REFERENCES

1. Gelman A E, Fisher A J, Huang H J, Baz M A, Shaver C M, Egan™, Mulligan M S. Report of the ISHLT working group on primary lung graft dysfunction Part III: Mechanisms: A 2016 consensus group statement of the International Society for Heart and Lung Transplantation. J Heart Lung Transplant 2017; 36(10):1114-1120. Doi:10.1016/j.healun.2017.07.014
2. Laubach V E, Sharma A K. Mechanisms of lung ischemia-reperfusion injury. Curr Opin Organ Transplant 2016; 21(3):246-252. Doi:10.1097/MOT.0000000000000304
3. Shah R J, Diamond J M. Primary Graft Dysfunction (PGD) Following Lung Transplantation. Semin Respir Crit Care Med 2018; 39(2):148-154. Doi:10.1055/s-0037-1615797
4. Christie J D, Kotloff R M, Ahya V N, Tino G, Pochettino A. Gaughan C, et al. The effect of primary graft dysfunction on survival after lung transplantation. Am J Respir Crit Care Med 2005:171(11):1312-1316. Doi:10.1164/rccm.200409-12430C
5. Daud S A, Yusen R D, Meyers B F, Chakinala M M, Walter M J, Aloush A A, et al. Impact of immediate primary lung allograft dysfunction on bronchiolitis obliterans syndrome. Am J Respir Crit Care Med 2007; 175(5):507-513. Doi:10.1164/rccm.200608-10790C
6. Daoud D, Chacon Alberty L, Wei Q, Hochman Mendez C, Virk M H M, Mase J, et al. Incidence of primary graft dysfunction is higher according to the new ISHLT 2016 guidelines and correlates with clinical and molecular risk factors. J Thorac Dis 2021; 13(6):3426-3442. Doi: 10.21037/jtd-20-3564
7. Haywood N, Ta H Q, Zhang A, Charles E J, Rotar E, Noona St, et al. Endothelial transient receptor potential vanilloid 4 channels mediate lung ischemia-reperfusion injury. Ann Thorac Surg 2022; 113(4):1256-1264. Doi:10.1016/j.athoracsur.2021.04.052
8. Soni H, Peixoto-Neves D. Olushoga M A, Adebiyi A. Pharmacological inhibition of TRPV4 channels protects against ischemia-reperfusion-induced renal insufficiency in neonatal pigs. Clin Sci (Lond) 2019; 133(9):CS20180815. Doi:10.1042/CS20180815
9. Tanaka K, Matsumoto S, Yamada T, Yamasaki R, Suzuki M, Kido M A, Kira J I. Reduced post-ischemic brain injury in transient receptor potential vanilloid 4 knockout mice. Front Neurosci 2020; 14:453. Doi:10.3389/fnins.2020.00453
10. Wang B, Wu Q, Liao J, Zhang S, Liu H, Yang C, et al. Propofol induces cardioprotection against ischemia-reperfusion injury via suppression of transient receptor potential vanilloid 4 channel. Front Pharmacol 2019; 10:1150. Doi: 10.3389/fphar.2019.01150
11. Sonkusare S K. Laubach V E. Endothelial TRPV4 channels in lung edema and injury. Curr Top Membr 2022:89:43-62. Doi:10.1016/bs.ctm.2022.07.001
12. Hamanaka K, Jian M Y, Weber D S, Alvarez D F, Townsley M I, Al-Mehdi A B, et al. TRPV4 initiates the acute calcium-dependent permeability increase during ventilator-induced lung injury in isolated mouse lungs. Am J Phvsiol Lung Cell Mol Physiol 2007; 293(4):L923-932. Doi:10.1152/ajplung.00221.2007
13. Haywood N, Ta H Q, Rotar E, Daneva Z, Sonkusare S K, Laubach V E. Role of the purinergic signaling network in lung ischemia-reperfusion injury. Curr Opin Organ Transplant 2021; 26(2):250-257. Doi:10.1097/MOT.0000000000000854
14. Scheraga R G, Southern B D, Grove L M. Olman M A. The role of TRPV4 in regulating innate immune cell function in lung inflammation. Front Immunol 2020:11:1211. Doi:10.3389/fimmu.2020.01211
15. Kuppusamy M, Ta H Q, Davenport H N, Bazaz A, Kulshrestha A, Daneva Z, et al. Purinergic P2Y2 receptor-induced activation of endothelial TRPV4 channels mediates lung ischemia-reperfusion injury. Sci Signal 2023; 16(808):eadg1553. Doi:10.1126/scisignal.adg1553
16. Cheung M. Bao W, Behm D J, Brooks C A, Bury M J, Dowdell S E, et al. Discovery of GSK2193874: An orally active, potent, and selective blocker of transient receptor potential vanilloid 4. ACS Med Chem Lett 2017; 8(5):549-554. Doi:10.1021/acsmedchemlett.7b00094
17. Thorneloe K S, Cheung M, Bao W, Alsaid H, Lenhard S, Jian M Y, et al. An orally active TRPV4 channel blocker prevents and resolves pulmonary edema induced by heart failure. Sci Transl Med 2012; 4(159):159ra148. Doi:10.1126/scitranslmed.3004276
18. LaPar D J, Laubach V E, Emaminia A, Crosby I K, Hajzus V A, Sharma A K, et al. Pretreatment strategy with adenosine A2A receptor agonist attenuates reperfusion injury in a preclinical porcine lung transplantation model. J Thorac Cardiovasc Surg 2011; 142(4):887-894. Doi:10.1016/j.jtcvs.2011.06.015
19. Gazoni L M, Laubach V E, Mulloy D P, Bellizzi A, Unger E B, Linden J, et al. Additive protection against lung ischemia-reperfusion injury by adenosine A2A receptor activation before procurement and during reperfusion. J Thorac Cardiovasc Surg 2008; 135(1):156-165. Doi:10.1016/j.jtcvs.2007.08.041
20. Sharma A K. Charles E J, Zhao Y, Narahari A K, Baderdinni P K, Good M E, et al. Pannexin-1 channels on endothelial cells mediate vascular inflammation during lung ischemia-reperfusion injury. Am J Physiol Lung Cell Mol Physiol 2018; 315(2):L301-L312. Doi:10.1152/ajplung.00004.2018
21. Giddings J C. Soluble adhesion molecules in inflammatory and vascular diseases. Biochem Soc Trans 2005; 33(Pt 2):406-408. Doi:I0.1042/BST0330406
22. Hashimoto K, Cypel M, Kim H, Machuca T N, Nakajima D, Chen M, et al. Soluble adhesion molecules during ex vivo lung perfusion are associated with posttransplant primary graft dysfunction. Am J Transplant 2017; 17(5):1396-1404. Doi:10.1111/ajt.14160
23. Pottecher J, Roche A C, Degot T, Helms 0, Hentz J G, Schmitt J P, et al. Increased extravascular lung water and plasma biomarkers of acute lung injury precede oxygenation impairment in primary graft dysfunction after lung transplantation. Transplantation 2017; 101(1):112-121. Doi:10.1097/TP.0000000000001434
24. Sakamaki F, Ishizaka A. Handa M. Fujishima S. Urano T. Sayama K, et al. Soluble form of P-selectin in plasma is elevated in acute lung injury. Am J Respir Crit Care Med 1995:151(6):1821-1826. Doi:10.1164/ajrccm.151.6.7539327
25. Jarvisalo M J, Juonala M, Raitakari O T. Assessment of inflammatory markers and endothelial function. Curr Opin Clin Nutr Metab Care 2006; 9(5):547-552. Doi:10.1097/01.mco.0000241663.00267.ae
26. Lee P H, Park S, Lee Y G, Choi S M, An M H, Jang A S. The impact of environmental pollutants on barrier dysfunction in respiratory disease. Allergy Asthma Immunol Res 2021; 13(6):850-862. Doi:10.4168/aair.2021.13.6.850
27. Assimakopoulos S F, Akinosoglou K, de Lastic A L, Skintzi A, Mouzaki A, Gogos C A. The prognostic value of endotoxemia and intestinal barrier biomarker ZO-1 in bacteremic sepsis. Am J Med Sci 2020; 359(2):100-107. Doi: 10.1016/j.amjms.2019.10.006
28. Kempuraj D. Aenlle K K, Cohen J, Mathew A, Isler D. Pangeni R P, et al. COVID-19 and long COVID: Disruption of the neurovascular unit, blood-brain barrier, and tight junctions. Neuroscientist 2023 Sep. 11; 10738584231194927 (Online ahead of print). Doi:10.1177/10738584231194927
29. Olsson A, Gustavsen S, Langkilde A R, Hansen T H, Sellebjerg F, Bach Sondergaard H, Oturai A B. Circulating levels of tight junction proteins in multiple sclerosis: Association with inflammation and disease activity before and after disease modifying therapy. Mult Scler Relat Disord 2021; 54:103136. Doi:10.1016/j.msard.2021.103136
30. Andersson E A, Mallard C, Ek C J. Circulating tight-junction proteins are potential biomarkers for blood-brain barrier function in a model of neonatal hypoxic/ischemic brain injury. Fluids Barriers CNS 2021; 18(1):7. Doi:10.1186/s12987-021-00240-9
31. Hendrickson C M, Matthay M A. Endothelial biomarkers in human sepsis: pathogenesis and prognosis for ARDS. Pulm Circ 2018; 8(2):2045894018769876. Doi: 10.1177/2045894018769876
32. Wang M, Gauthier A, Daley L, Dial K, Wu J, Woo J, et al. The role of HMGB1, a nuclear damage-associated molecular pattern molecule, in the pathogenesis of lung diseases. Antioxid Redox Signal 2019; 31(13):954-993. Doi:10.1089/ars.2019.7818
33. Sharma A K, LaPar D J, Stone M L, Zhao Y, Kron I L, Laubach V E. Receptor for advanced glycation end products (RAGE) on iNKT cells mediates lung ischemia-reperfusion injury. Am J Transplant 2013; 13(9):2255-2267. Doi:10.1111/ajt.12368
34. Wolfson R K, Chiang E T, Garcia J G. HMGB1 induces human lung endothelial cell cytoskeletal rearrangement and barrier disruption. Microvasc Res 2011; 81(2):189-197. Doi: 10.1016/j.mvr.2010.11.010
35. Goyal N, Skrdla P, Schroyer R, Kumar S, Fernando D. Oughton A, et al. Clinical Pharmacokinetics, Safety, and Tolerability of a Novel, First-in-Class TRPV4 Ion Channel Inhibitor. GSK2798745. in Healthy and Heart Failure Subjects. Am J Cardiovasc Drugs 2019; 19(3):335-342. Doi: 10.1007/s40256-018-00320-6
36. Daneva Z, Marziano C, Ottolini M, Chen Y L, Baker™, Kuppusamy M. et al. Caveolar peroxynitrite formation impairs endothelial TRPV4 channels and elevates pulmonary arterial pressure in pulmonary hypertension. Proc Natl Acad Sci USA 2021; 118(17):e2023130118. Doi:10.1073/pnas.2023130118
37. Hong K, Cope E L, DeLalio L J, Marziano C, Isakson B E, Sonkusare S K. TRPV4 (Transient Receptor Potential Vanilloid 4) Channel-Dependent Negative Feedback Mechanism Regulates G(q) Protein-Coupled Receptor-Induced Vasoconstriction. Arterioscler Thromb Vasc Biol 2018; 38(3):542-554. Doi: 10.1161/ATVBAHA.117.310038
38. Pairet N, Mang S, Fois G, Keck M, Kuhnbach M. Gindele J, et al. TRPV4 inhibition attenuates stretch-induced inflammatory cellular responses and lung barrier dysfunction during mechanical ventilation. PloS One 2018; 13(4):e0196055. Doi:10.1371/journal.pone.0196055

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

What is claimed:

1. A method for treating lung ischemia-reperfusion injury (IRI) in a subject in need thereof, the method comprising administering to the subject a P2Y2 receptor (P2Y2R) inhibitor.

2. The method of claim 1, wherein the P2Y2R inhibitor inhibits at least one of P2Y2R expression and activity.

3. The method of claim 2, wherein the P2Y2R inhibitor inhibits P2Y2R activity.

4. The method of claim 1, wherein the P2Y2R inhibitor is a selected from a compound, a protein, and an inhibitory nucleic acid.

5. The method of claim 4, wherein the P2Y2R inhibitor is a compound.

6. The method of claim 5, wherein the compound is AR-C 118925XX (ARC) or a pharmaceutically acceptable salt thereof.

7. The method of claim 4, wherein the inhibitory nucleic acid is selected from a dsRNA, a siRNA, a shRNA, a miRNA, an ami-RNA, an antisense oligonucleotide (ASO), and an aptamer.

8. The method of claim 4, wherein the protein is an antibody.

9. The method of claim 1, wherein the subject has undergone or will undergo a lung transplantation.

10. The method of claim 1, wherein the method further comprises performing a lung transplantation in the subject.

11. The method of claim 9, wherein the P2Y2R inhibitor is administered before the lung transplantation.

12. The method of claim 9, wherein the P2Y2R inhibitor is administered at the same time as the lung transplantation.

13. The method of claim 9, wherein the P2Y2R inhibitor is administered after the lung transplantation.

14. The method of claim 13, wherein the P2Y2R inhibitor is administered between about one hour before the lung transplantation and about one hour after the lung transplantation procedure.

15. The method of claim 1, wherein the P2Y2R inhibitor is administered intravenously.

16. The method of claim 1, further comprising administering to the subject at least one of a TRPV4 inhibitor and a Panx1 inhibitor.

17. The method of claim 16, wherein each of the at least one TRPV4 inhibitor and Panx1 inhibitor is selected from a compound, a protein, and an inhibitory nucleic acid.

18. The method of claim 17, wherein the TRPV4 inhibitor is GSK2193874.

19. The method of claim 17, wherein the Panx1 inhibitor is spironolactone.

20. The method of claim 16, wherein the TRPV4 inhibitor and the Panx1 inhibitor are administered at the same time as the P2Y2R inhibitor.


SEQUENCE LISTING (9 sequences)

SEQ ID NO: 1 - DNA, 26 bp, synthetic construct
  source 1..26: mol_type = other DNA
gcctgcattaccggtcgatgcaacga

SEQ ID NO: 2 - DNA, 26 bp, synthetic construct
  source 1..26: mol_type = other DNA
gtggcagatggcgcggcaacaccatt

SEQ ID NO: 3 - DNA, 24 bp, synthetic construct
  source 1..24: mol_type = other DNA
catgaaatctgacctcttgtcccc

SEQ ID NO: 4 - DNA, 24 bp, synthetic construct
  source 1..24: mol_type = other DNA
cggaccacacgtctgtcatgtgtt

SEQ ID NO: 5 - DNA, 22 bp, synthetic construct
  source 1..22: mol_type = other DNA
tcccaactcacacgtacaaatg

SEQ ID NO: 6 - DNA, 28 bp, synthetic construct
  source 1..28: mol_type = other DNA
caacaagtttgtacaaaaaagcaggctg

SEQ ID NO: 7 - DNA, 28 bp, synthetic construct
  source 1..28: mol_type = other DNA
tgcttgtatagatatctgacccagagct

SEQ ID NO: 8 - DNA, 18 bp, synthetic construct
  source 1..18: mol_type = other DNA
cccagctttcttgtacaa

SEQ ID NO: 9 - DNA, 20 bp, synthetic construct
  source 1..20: mol_type = other DNA
tgacgactcaagacggacag